(19) United States
(12) Patent Application Publication
(10) Pub. No.: US 2021/0255187 A1
(43) Pub. Date: Aug. 19, 2021

(54) METHOD FOR PREDICTING THE EFFECTIVENESS OF TREATMENTS FOR CANCER PATIENTS

(71) Applicant: University College Dublin, National University of Ireland, Dublin, Belfield (IE)

(72) Inventor: Dirk Fey, Dublin (IE)

(21) Appl. No.: 17/243,373

(22) Filed: Apr. 28, 2021

Related U.S. Application Data
(63) Continuation of application No. PCT/EP2019/079473, filed on Oct. 29, 2019.

(30) Foreign Application Priority Data
Oct. 29, 2018 (GB) 1817651.1

(51) Int. Cl. G01N 33/574; G16H 50/20

(52) U.S. Cl. CPC G01N 33/574; G01N 2800/60; G01N 2800/52; G16H 50/20

(57) ABSTRACT

A method for predicting whether a patient with cancer is likely to respond to a treatment is described. The method comprises measuring a value indicative of a level of certain biomarkers; calculating a total value using a weighted sum of the measured values; comparing the total value to a threshold value and when the total value is below the threshold value, determining that the patient is likely to respond to the treatment. Alternatively, the method comprises applying a model comprising coupled ordinary differential equations defining the rate of change of a plurality of biomarkers to predict a plurality of output values for the biomarkers for the treatment; selecting a biomarker; comparing the output value for the selected biomarker to an associated threshold value for the selected biomarker; and when the output value is below the associated threshold value, determining that the patient is likely to respond to the treatment.

### CROSS-REFERENCES TO RELATED APPLICATIONS

This application is a continuation application of International Patent Application No. PCT/EP2019/079473 entitled “METHOD FOR PREDICTING THE EFFECTIVENESS OF TREATMENTS FOR CANCER PATIENTS,” filed on Oct. 29, 2019, which claims priority to Great Britain Patent Application No. 1817651.1, filed on Oct. 29, 2018, all of which are herein incorporated by reference in their entirety for all purposes.

### FIELD

The present invention relates to a method for predicting the effectiveness of treatments such as chemotherapy for cancer patients and personalising drug-response predictions, for example for neuroblastoma, breast cancer, lung adenocarcinoma, kidney renal clear cell carcinoma and liver hepatocellular carcinoma.

### BACKGROUND

Neuroblastoma is one of the most common cancers in young children. Its course can be very different from rather benign to highly aggressive. Despite recent advances in immunotherapies, the mainstay of treatment is genotoxic chemotherapy which causes severe long-term side effects in 60 to 90% of survivors.

As a method for classifying a patient's disease state, biomarkers are cornerstones of clinical medicine. Personalized medicine, in particular, is highly dependent on reliable and highly accurate biomarkers for individual diagnosis and treatment choice. Although biomarkers have been invaluable tools in the arsenal of clinical diagnostics, by design, biomarkers, including multi-omic signatures, can only provide a snapshot of a patient's disease state. There are a number of limitations. For example, biomarkers cannot accurately assess a patient's disease and drug-response mechanisms, be used to simulate the intracellular changes triggered by drug treatments, anticipate/predict the development of drug resistance due to these intracellular changes or integrate mechanistic biological knowledge into the prediction or clinical decision.

As set out in the paper entitled “Two-phase dynamics of p53 in the DNA damage response” by Zhang et al published in W. PNAS 108, 8990-8995 (2011), it can be useful to study the tumour suppressor p53 as a clue to cancer treatment. In unstressed cells, p53 is kept at low levels by its negative regular MDM2. Upon DNA damage, p53 is stabilized and activated which may lead to cell cycle arrest and/or apoptosis. Cell cycle arrest may facilitate DNA repair and promote cell survival. Apoptosis may provide an efficient way to remove irreparably damaged cells. In the paper, a model of the p53 network to clarify the link between p53 dynamics and DNA damage response (DDR) is proposed which uses four modules: a DNA repair module, an ATM sensor, a p53-centered feedback control module and a cell fate decision module.

Another paper which considers biological mechanisms is “Signaling pathway models as biomarkers: Patient-specific simulations of JNK activity predict the survival of neuroblastoma patients” by Fey et al. published in Science Signaling 8(408), ra130 (2015) which explains that the dynamic states of cancer cell signalling are intimately linked to clinical outcomes. In the proposed method, patient-specific simulations are generated by adjusting the total protein concentration of each model component to the measured values from a patient's tumour sample.

In order to reduce side effects and improve therapeutic efficacy, the present applicant has recognised that improved biomarkers and personalised methods for predicting chemotherapy are desperately needed.

### BRIEF SUMMARY

According to the present invention there is provided an apparatus and method as set forth in the appended claims. Other features of the invention will be apparent from the dependent claims, and the description which follows.

We describe a method for predicting whether a patient with cancer is likely to respond to a particular treatment, the method comprising measuring a value indicative of a level of each of the biomarkers ATM, CHEK2, TP53, MDM2, PPM1D, SIAH1, HIPK2 and WSB1 within a sample from the patient; calculating a total value using a weighted sum of the measured values; wherein each biomarker value has an associated weight; comparing the total value to a threshold value and when it is determined that the total value is below the threshold value, determining that the patient is likely to respond to the treatment. The biomarkers ATM, CHEK2, TP53, MDM2, PPM1D, SIAH1, HIPK2 and WSB1 may be considered to form a panel of biomarkers.

In this method, the sample may be a tumour sample and may be collected from a patient using any known technique. The biomarkers may be a set of genes, proteins, and/or any of their paralogs, isoforms, or genes with identical or similar biological functions, for example as provided in the following table 1. Together the biomarkers may be considered to be a panel of biomarkers. For each gene, the gene ID number is provided. This is the unique identifier number as assigned by the National Center for Biotechnology Information, Gene database (https://www.ncbi.nlm.nih.gov/gene) or Entrez database (https://www.ncbi.nlm.nih.gov/class/MLACourse/Orginal8Hour/Entrez). For each protein, the unique entry number as determined by the UniProtKB database (https://www.uniprot.org/uniprot/) is provided.

TABLE 1

| | | | | Paralog / | | Similar | |
|---|---|---|---|---|---|---|---|
| Gene | Entrez | Protein | UniProtKB | Similar gene | Entrez | protein | UniProtKB |
| ATM | 472 | ATM | Q13315 | ATR | 545 | ATR | Q13535 |
| CHEK2 | 11200 | CHK2 | O96017 | CHEK1 | 1111 | CHK1 | O14757 |
| TP53 | 7157 | p53 | P04637 | TP63, TP73 | 8626, 7161 | p63, p73 | Q9H3D4 |
| | | | | | | | O15350 |
| MDM2 | 4193 | MDM2 | Q00987 | MDM4 | 4194 | MDMX | O15151 |
| PPM1D | 8493 | WIP1 | O15297 | | | | |
| SIAH1 | 6477 | SIAH1 | Q8IUQ4 | WSB1, SIAH2 | 26118, 6478 | WSB1, SIAH2 | Q9Y6I7, O43255 |
| HIPK2 | 28996 | HIPK2 | Q9H2X6 | HIPK1 | 204851 | HIPK1 | Q86Z02 |

A set of training data may be used to determine the associated weights for each biomarker value. Any suitable analysis method may be used to determine the associated weights, for example a Cox regression analysis may be used to determine the associated weights.

Where appropriate, the method may be adapted to be a method of treatment. The method may thus comprise applying the treatment when it is determined that the patient is likely to respond to the treatment. When it is determined that the total value is equal to or above the threshold, the method may further comprise determining that the patient is not likely to respond to the treatment and determining an alternative treatment. For example, the treatment used in the prediction may be chemotherapy and when it is determined that this is not a suitable treatment, an alternative treatment, for instance immunotherapy with GD2 targeting drugs such as dinutuximab (Unituxin), may be provided.

The cancer may be neuroblastoma. For example, the chemotherapy treatment may be induction chemotherapy which may use alternating regimens of several drugs (typically cisplatin, etoposide, vincristine, cyclophosphamide, doxorubicin and topotecan. Alternatively, the cancer may be breast cancer. Further, because the method relies on the molecular characteristics of the cancer, not its tissue of origin, the method may be applied to any patient with a p53 wild-type cancer that does not harbour a p53 mutation. Alternatively, the cancer may be lung adenocarcinoma, kidney renal clear cell carcinoma and liver hepatocellular carcinoma We also describe the use of ATM, CHEK2, TP53, MDM2, PPM1D, SIAH1, HIPK2 and WSB1 as biomarkers in a method for predicting whether a patient with cancer is likely to respond to a particular treatment, e.g. chemotherapy as described above. We also describe a kit comprising reagents that specifically bind to each member of a panel of biomarkers consisting of ATM, CHEK2, TP53, MDM2, PPM1D, SIAH1, HIPK2 and WSB1 or their proteins, and/or any of their paralogs, isoforms, or genes with similar biological functions as provided in the table above. The reagents may be PCR primer sets. We also describe use of the kit in the method described above.

We also describe a method for predicting whether a patient with cancer is likely to respond to a particular treatment, the method comprising: applying a model comprising a set of coupled ordinary differential equations defining the rate of change of a plurality of biomarkers to predict a plurality of output values for each of the biomarkers for the treatment; selecting a biomarker from the plurality of biomarkers; comparing the output value for the selected biomarker to an associated threshold value for the selected biomarker; and when the output value for the selected biomarker is below the associated threshold value, determining that the patient is likely to respond to the treatment; wherein the plurality of biomarkers include p53, ATM, CHK2, SIAH1, HIPK2, WIP1 and MDM2.

In this method, the sample may be a tumour sample. The biomarkers may be a set of proteins. Genes codes for proteins that perform certain functions within a network. The model may thus be considered to be predicting the functional activity of the proteins or predicting the network activity of these proteins as explained in more detail below.

A set of training data may be used to determine the associated weights for each biomarker value. Any suitable analysis method may be used to determine the associated weights, for example a Cox regression analysis may be used to determine the associated weights.

The plurality of biomarkers may comprise unphosphorylated and phosphorylated forms of at least one of ATM, p53, SIAH1, WSB1, CHK1 and CHK2. The phosphorylated forms of p53 may include pro-apoptotic residues such as S46 and cell-cycle arrest residues such as S15. The plurality of biomarkers may comprise mRNA amounts for at least one of p53, WIP1, MDM2, MDM4 and MDMX. The plurality of biomarkers comprise protein amounts for at least one of HIPK2, WIP1, MDM2, MDM4 and MDMX.

The biomarker may be selected from a phosphorylated form of p53 at cell-cycle arrest residues such as S15, a phosphorylated form of ATM, a phosphorylated form of CHK2, a phosphorylated form of SIAH1, HIPK2, WIP1 and MDM2. The method may further comprise comparing at least one of a peak value for the phosphorylated form of p53 at cell-cycle arrest residues such as S15, a peak value for the phosphorylated form of ATM, a peak value for the phosphorylated form of CHK2, a peak value for the phosphorylated form of SIAH1, a half activation value for HIPK2, a half activation value for WIP1 and a half activation value for MDM2, an amplitude value for HIPK2, an amplitude value for WIP1 and an amplitude value for MDM2.

Applying the model may comprise predicting at least one of a peak value, an amplitude value and a half-activation value for each biomarker.

Where appropriate, the method may be adapted to be a method of treatment. The method may thus comprise applying the treatment when it is determined that the patient is likely to respond to the treatment. The method may further comprise when it is determined that the total value is equal to or above the threshold, determining that the patient is not likely to respond to the treatment and determining an alternative treatment. For example, the treatment used in the prediction may be chemotherapy and when it is determined that this is not a suitable treatment, an alternative treatment, for instance immunotherapy with GD2 targeting drugs such as dinutuximab (Unituxin), may be provided.

The cancer may be neuroblastoma. Alternatively, the cancer may be breast cancer, lung adenocarcinoma, kidney renal clear cell carcinoma and liver hepatocellular carcinoma.

A set of training data may be used to determine the associated thresholds for each biomarker value. A set of training data may be used to determine any parameters within the model. For example, a Cox regression analysis may be used to determine the associated thresholds and/or parameters.

The method may be personalised to a patient. For example, the method may further comprises providing a sample (e.g. a tumour sample) from the patient; measuring a value indicative of a level of each of the biomarkers ATM, CHEK2, TP53, MDM2, PPM1D, SIAH1, HIPK2 and WSB1 within the sample; personalising the model to the patient by incorporating the measured values. The gene expression (e.g. mRNA measurements) may be used for each of the biomarkers. Genes code for proteins that perform certain functions within a network. By including the proteins as the biomarkers and measuring gene expression, e.g. by measuring mRNA levels, the model may be thus defined as a model to predict the functional activity of these proteins. In this way, the model may be providing an understanding of what the genes associated with these proteins do. Alternatively, one can also measure the protein expression (protein concentration). In this way, the method predicts the (network) activity of these proteins. Any combination of gene and protein expression may be measured. Different equations apply for using gene and protein expression as detailed in the tables below.

Personalising the model may comprise defining patient-specific parameters for each of the measure biomarkers. For example, the patient-specific parameters may comprise at least one of a value, ATMtot, for the gene or protein expression of ATM, a value, CHK2tot, for the gene or protein expression of CHK2, a value, SIAH1tot, for the gene or protein expression of either of SIAH1 and WSB1, a basal synthesis parameter, ksp530, for p53 mRNA, a basal synthesis parameter, ksmdm20, for MDM2 mRNA, a basal synthesis parameter, kswip10, for WIP1 and a rate parameter, kshipk2, for HIPK2 translation.

We also describe a method for predicting whether a patient with cancer is likely to respond to a particular treatment, the method comprising: applying a model comprising a set of coupled ordinary differential equations defining the rate of change of a plurality of biomarkers to calculate a first output value for a biomarker in the plurality of biomarkers, wherein the model comprises a plurality of parameter values associated with the plurality of biomarkers; selecting another biomarker from the plurality of biomarkers; selecting a treatment which targets the selected biomarker; perturbing the parameter value corresponding to the selected biomarker in the model, applying the model using the perturbed parameter value to calculate a second output value for the biomarker, comparing the first and second output values to derive a sensitivity value for the selected biomarker; iterating the selecting and calculating steps for further biomarkers to calculate a plurality of sensitivity values; and identifying the selected biomarker having a largest sensitivity value in the plurality of sensitivity values.

The sensitivity value may be derived by calculating at least one of the difference and the ratio between the first and second output values. The first and second output values may be a peak value, an amplitude value and/or a half-activation value. For each the first and second values may be any one or a combination of the peak value for the phosphorylated form of p53 at cell-cycle arrest residues such as S15, a peak value for the phosphorylated form of ATM, a peak value for the phosphorylated form of CHK2, a peak value for the phosphorylated form of SIAH1, a half activation value for HIPK2, a half activation value for WIP1 and a half activation value for MDM2, an amplitude value for HIPK2, an amplitude value for WIP1 and an amplitude value for MDM2.

Where appropriate, the method may be adapted to be a method of treatment. The method may thus comprise applying the treatment which targets the identified biomarker. Such a method may thus be used to identify the best patient-specific treatment in form of a targeted drug that targets any of the genes/proteins in the model. Alternatively, the treatment which may be identified may be anti-cancer treatment, e.g. one or more of chemotherapy, radiotherapy, surgery or immunotherapy. The model can simulate the impact of any drug or treatment targeting any gene in the model, that is the sensitivity figure, and then identify the most effective one. This also works for identifying optimal personalised drug-combinations, e.g. combining a DNA-damaging chemo drug with a targeted drug against one of the genes/proteins in the model.

We also describe a kit comprising reagents that specifically bind to each member of a panel of biomarkers consisting of ATM, CHEK2, TP53, MDM2, PPM1D, SIAH1, HIPK2 and WSB1 or their proteins. The reagents may be PCR primer sets. We also describe use of the kit in the method described above.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a better understanding of the invention, and to show how embodiments of the same may be carried into effect, reference will now be made, by way of example only, to the accompanying diagrammatic drawings in which:

FIG. 1A is a schematic block diagram showing the components of the system;

FIG. 1B is a flowchart showing a method which may be carried out using the system of FIG. 1A;

FIG. 2 is a schematic model used in the system;

FIG. 3A shows representative Western blots for various markers in a 10 hour window treated with different doses of doxorubicin in SH-SY5Y neuroblastoma cells;

FIG. 3B shows representative Western blots for the markers in FIG. 3A varying with time for a fixed dose of doxorubicin;

FIGS. 3C to 3F plot the normalised variation against time in both the measured and modelled data for the markers ATMp, p53s15tot, p53s46 and p53tot from FIG. 3A;

FIGS. 3G to 3J plot the normalised variation against dosage in both the measured and modelled data for the markers ATMp, p53s15tot, p53s46 and p53tot respectively;

FIGS. 4A to 4F plot the normalised variation against time in both measured and modelled data for MCF7 breast cancer cells for the markers ATMp, CHK2p, p53tot, MDM2m, WIP1m and WIP1 respectively;

FIG. 5 shows tumour and clinical data from 688 neuroblastoma patients;

FIGS. 6A and 6B plot examples of the simulated output against dosage for the peaked outputs of p53s15 and sigmoidal model output for p53s46 respectively;

FIG. 6C is a flowchart showing a method which may be carried out using the system of FIG. 1A and the model of FIG. 2;

FIGS. 7A and 7B plot the eventfree survival for patients against time for patients stratified into two groups using the peak and last values for p53s15 respectively;

FIGS. 8A and 8B plot the eventfree survival for patients against time using the peak and last values for p53s46 respectively;

FIGS. 9A to 9C stratify low risk, intermediate risk and high risk cohorts in to two groups using the peak value of p53s15;

FIGS. 10A to 10C stratify low risk, intermediate risk and high risk cohorts in to two groups using the half-activation threshold of CHK2p;

FIGS. 11A to 11C plot the hazard ratios for different systems for stratifying the entire cohort, the high risk cohort and the low risk cohort, respectively;

FIGS. 11D and 11E plot the eventfree survival for patients against time for patients stratified into two groups using two variants of the data only model of FIGS. 11A to 11C;

FIGS. 12A and 12B show the sensitivities for various genes and proteins in the models for the half-activation threshold of CHK2p and peak of p53s15 respectively;

FIG. 12C shows another method which may be performed on the system of FIG. 1A;

FIGS. 13A and 13B plot the overall survival for patients having breast cancer against time using the peak values of p53s15 and p53s46, respectively;

FIGS. 14A and 14B plot the overall survival for patients having lung cancer against time using the peak values of p53s15 and p53s46, respectively;

FIGS. 15A and 15B plot the overall survival for patients having kidney renal clear cell carcinoma against time using the peak values of p53s15 and p53s46, respectively; and

FIGS. 16A and 16B plot the overall survival for patients having liver cancer against time using the peak values of p53s15 and p53s46, respectively.

### DETAILED DESCRIPTION

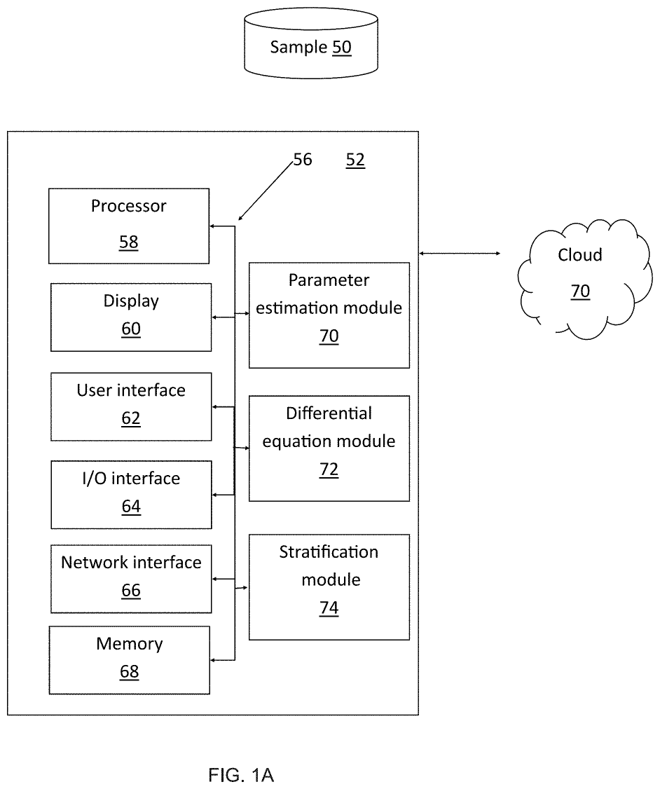

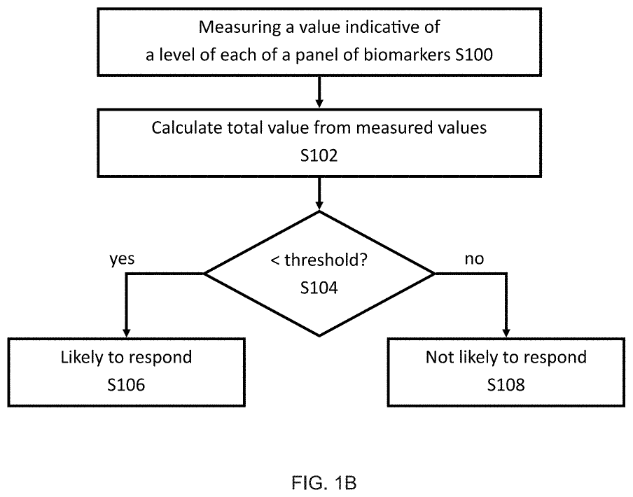

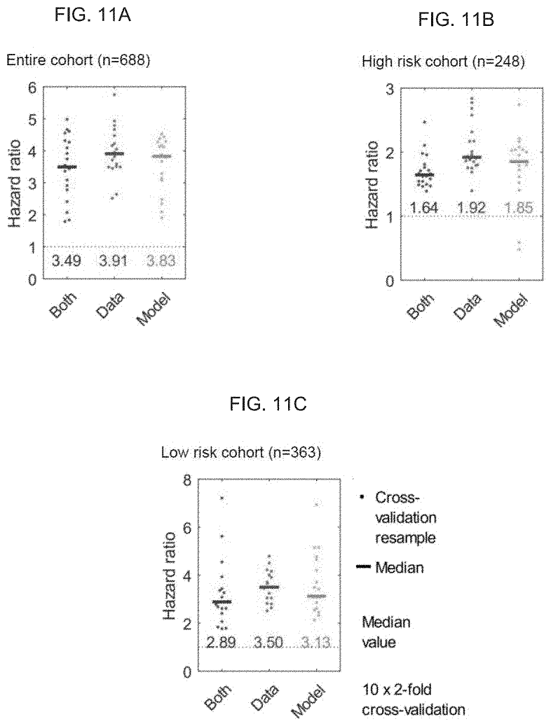

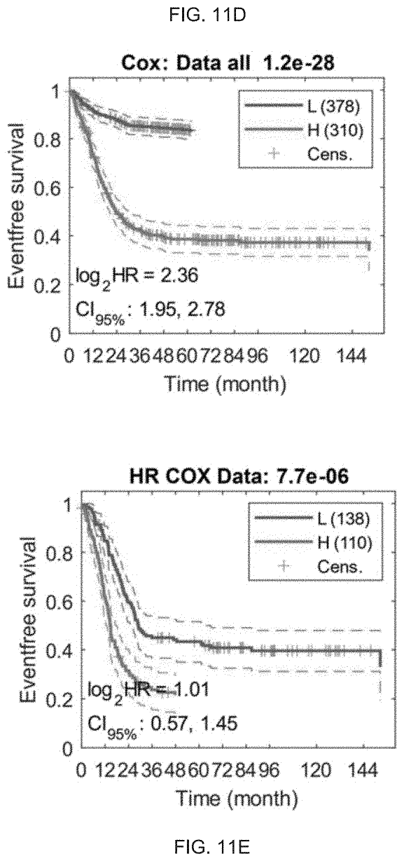

FIG. 1A shows the various components for implementing the system and FIG. 1B shows the steps which may be carried out. A tumour sample 50 from a patient may be treated and the values of various components, e.g. levels of each of a panel of biomarkers, within the sample 50 may be measured as described in more detail below and shown in step S100. These values may be input into a prognosis system 52 by a user inputting the data through a user interface 62 or via an input/output (I/O) interface(s) 64 which may facilitate the receipt of the tumour data from one or more I/O devices. The prognosis system may be formed from one or more servers and may comprise one or more processors 58 for processing the received measurements and modelling patient outcomes as described below. For example, the total value may be calculated from the measured values (step S102), e.g. using a weighted sum as described in more detail below—see FIGS. 11A to 11C for one example. Alternatively, as shown schematically, the prognosis system may be coupled to the cloud 71 via a network interface 66 so that some or all of the processing can take in the cloud. The network interface(s) 66 may enable communication via one or more networks, e.g. the internet.

The prognosis system may also comprise memory 68 and one or more buses 56 that functionally couple the various components/modules. The bus(es) 56 may be any suitable bus, including a system bus, a memory bus, or an address bus and may be associated with any suitable bus architecture. The memory 68 may include volatile memory (memory that maintains its state when supplied with power) such as random access memory (RAM) and/or non-volatile memory (memory that maintains its state even when not supplied with power) such as read-only memory (ROM). The memory 68 may include main memory as well as various forms of cache memory. Computer-executable code, instructions, or the like that may be loadable into the memory 68 and are executable by the processor(s) 58 to cause the processor(s) 58 to perform or initiate various operations. Moreover, output data generated as a result of execution of the computer-executable instructions by the processor(s) 58 may be stored at least initially in memory 68.

The processor(s) 58 may include any type of suitable processing unit including both software and hardware. For example, the processor may be a central processing unit, a microprocessor, a Reduced Instruction Set Computer (RISC) microprocessor, a Complex Instruction Set Computer (CISC) microprocessor, a microcontroller, an Application Specific Integrated Circuit (ASIC), a Field-Programmable Gate Array (FPGA), a System-on-a-Chip (SoC), a digital signal processor (DSP), and so forth. The I/O interface(s) 64 may include an interface for an external peripheral device connection where such an external device can be used to generate the tumour data. For example, the connection may be via a wired connection, e.g. USB or similar connection protocols or via a wireless connection, including WiFi, radio or Bluetooth etc.

There may also be one or more modules for undertaking the various steps of the method. For example, there may be a parameter estimation module 70 which as explained in more detail below estimates the constant parameters in the model. The module may use a set of training data to generate a best fit of the parameters, e.g. using Cox regression analysis or a similar technique. There may also be a differential equation module 72. As explained in more detail below, the model is a set of coupled ordinary differential equations (ODE) that can be solved numerically (simulated) using ODE-solvers. There may also be a stratification module 74 which as explained below stratifies a group of patients into those likely to have a favourable prognosis and those likely to have a non-favourable prognosis. The stratification may be done by comparing the model generated value for a particular component (state) or calculated total value in the example of FIG. 1B with a threshold value for that component (step S104). If the calculated value is lower than the threshold, the patient may be considered to be likely to respond to the treatment (step S106) or if the calculated value is above the threshold the patient may be considered to be unlikely to respond to the treatment (step S108). It will also be appreciated that if more than one threshold is derived, stratification into more groups can be obtained.

It will be appreciated that these modules may include any combination of software, firmware, and/or hardware. The software and/or firmware may include computer-executable code, instructions, or the like that may be loaded into the memory for execution by one or more of the processor(s) to perform any of the operations described earlier in connection with correspondingly named modules. The parameters which are generated may also be stored in the memory or in a separate data storage which is accessible by the system.

It should be appreciated that the components and program modules depicted in FIG. 1A are merely illustrative and processing may alternatively be distributed across multiple modules, or the like, or performed by a different module, or the like. In addition, engines or program modules that support the functionality described herein may form part of one or more applications executable across any number of devices of the system in accordance with any suitable arrangement. In addition, any of the functionality described as being supported by any of the program modules may be implemented, at least partially, in hardware and/or firmware across any number of devices.

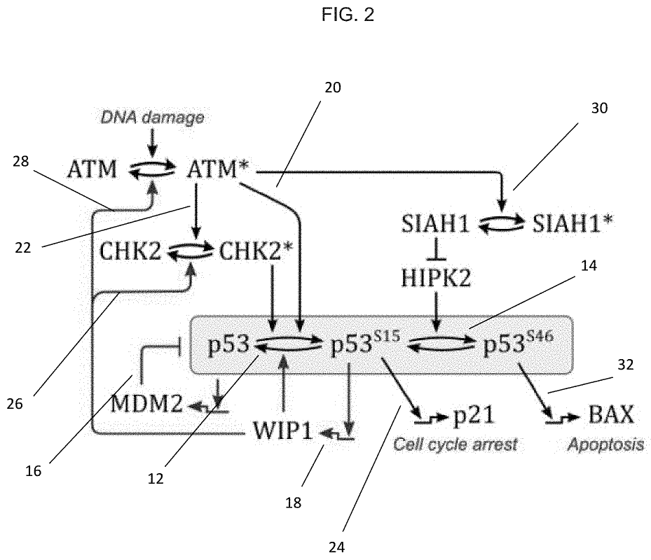

FIG. 2 shows the model which is used in the system and which describes the dynamics of p53, focussing on core components of the DNA damage response system. A full description of the model, including all reactions, equations and parameters is set out below. The model incorporates the core components of how DNA damage regulates p53 and its cell-cycle arrest and pro-apoptotic functions. The model features two feed-forward loops 12, 14 activating both the cell-cycle arrest and pro-apoptotic functions of p53 (as induced by p53 states denoted by S15 and S46, respectively, in the model), and two negative feedback loops 16, 18 via MDM2 and WIP1.

As shown schematically in the model of FIG. 2, DNA damage activates ATM, which can induce p53 phosphorylation of S15 directly via a first path 20 and indirectly via activation of the CHK2 kinase as indicated by path 22. As indicated by path 24, S15 phosphorylated p53 primarily induces cell cycle arrest via stimulating the expression of the p21 and other cell cycle genes, but it also induces the phosphatase WIP1 as indicated by the negative loop 18. This exerts multiple negative feedback loops 26, 28 by dephosphorylating p515 and both CHK2 and ATM. The transition of S15 to the proapoptotic S46 phosphorylation as shown in feedback loop 14 is promoted by the ATM inactivating the ubiquitin ligases SIAH1 and WSB1. This is modelled as a combined component that inhibits the S46 kinase HIPK2 as shown by path 30. S46 phosphorylated p53 induces expression of BAX and other pro-apoptotic genes as shown by path 32. In addition, feedback loop 16 includes the negative p53 autoregulation exerted by MDM2, which is primarily induced by both phosphorylated versions of p53 (S15 and S46), and in turn promotes p53 protein degradation. The decision between cell cycle arrest and apoptosis depends on the complex kinetic coordination of S15 and S46 phosphorylation of p53. The model thus comprises the following states which are also termed model components and the terms can be used interchangeably:

| State | Meaning |
|---|---|
| ATM | Unphosphorylated (inactive) form of ATM and/or ATR |
| ATMp | Phosphorylated (active) form of ATM and/or ATR |
| p53m | p53 mRNA |
| p53i | Unphosphorylated p53 |
| p53s15 | p53 phosphorylated at cell-cycle arrest residues such as S15 |
| p53s46 | p53 phosphorylated at pro-apoptotic residues such as S46 |
| SIAH1 | Combined pool of unphosphorylated SIAH1 and WSB1 |
| SIAH1p | Combined pool of phosphorylated SIAH1 and WSB1 |
| HIPK2 | HIPK2 protein |
| WIP1m | WIP1 mRNA |
| WIP1 | WIP1 protein |
| CHK2 | Unphosphorylated (inactive) CHK2 and/or CHK1 |
| CHK2p | Phosphorylated (active) CHK2 and/or CHK1 |
| MDM2m | MDM2 mRNA and or mRNA of MDM4 and MDMX |
| MDM2 | MDM2 protein and or protein of MDM4 and MDMX |

SIAH1, WSB1 and HIPK2 are not included in the Zhang model taught in “Two-phase dynamics of p53 in the DNA damage response” by Zhang et al published in W. PNAS 108, 8990-8995 (2011). The inclusion of these model components is important because these components mediate the pro-apoptotic signalling axes of p53 and contain important prognostic information. It is also noted that AKT which is included in the Zhang model, is omitted from the proposed model because data from breast cancer cells show that AKT phosphorylation is not changing in response to doxorubicin in breast cancer cells.

The proposed model focusses on modelling the genes and proteins for which experimental data is available. Increasing the number of components within the model increases its complexity and it is more difficult to calibrate the model (to estimate the parameters) which may thus result in decreased reliability of the model predictions. The proposed model is a useful model which is not too complex but is a reasonable predictor as evidenced below.

In the model, the states and their dynamic changes are described by ordinary differential equations, using the appropriate rate laws for each type of reaction. Such rate laws may be derived using known techniques, for example those taught in “Modelling with ordinary differential equations” by Fey et al published in Quantitative Biology: Theory, Computational Methods and Examples of Models, Vol. 1st edition (MIT Press, Cambridge, Mass., 2018).

The rates of change with time for each of the model components are set out below. Together, these equations describe a system of coupled ordinary differential equations (ODE) that can be solved numerically (simulated) using ODE-solvers, such as ode15s provided in MATLAB, a software tool for scientific computing. Many other solvers and software exist.

$$\frac{d}{d t} A T M = - v 1 + v 2 \\ \frac{d}{d t} A T {M}_{p} = v 1 - v 2 \\ \frac{d}{d t} p 53 m = v 12 - v 29 \\ \frac{d}{d t} p 5 {3}_{i} = - v 5 - v 6 + v 7 + v 21 - v 25 \\ \frac{d}{d t} p 5 {3}_{s 15} = v 5 + v 6 - v 7 - v 8 + v 9 - v 26 \\ \frac{d}{d t} p 5 {3}_{s 46} = v 8 - v 9 - v 27$$

$$\frac{d}{d t} SIAH 1 = - v 10 + v 11$$

$$\frac{d}{d t} S I A H {1}_{p} = v 10 - v 11$$

$$\frac{d}{d t} HIPK 2 = v22 - v28 - v 36$$

$$\frac{d}{d t} WIP 1 m = v 15 + v 16 - v 30$$

$$\frac{d}{d t} WIP 1 = v 23 - v 31$$

$$\frac{d}{d t} C H K 2 = - v 3 + v 4$$

$$\frac{d}{d t} C H K {2}_{p} = v 3 - v 4$$

$$\frac{d}{d t} M D M 2 m = v 13 + v 14 - v 32$$

$$\frac{d}{d t} M D M 2 = v 24 - v 33$$

$$\frac{d}{d t} p 21 m = v 17 + v 18 - v 34$$

$$\frac{d}{d t} B A X m = v 19 + v 20 - v 35$$

The reaction rates v1 to v35 are a convenient way to express the complex rate equations and the interaction between the different states. v1 to v35 are functions of the model states and parameters as defined below.

For example, v1 to v11 are equations relating to phosphorylation (activation) and dephosphorylation (deactivation) as defined below:

$$v 1 = k p a t m * A T M * D D R * \frac{k u}{j p a t m + A T M}$$

$$v2 = kdpatm * WIP 1 * \frac{A T {M}_{p}}{j d p a t m + A T {M}_{p}}$$

$$v 3 = k p c h k 2 * A T {M}_{p} * \frac{C H K 2}{j p c h k 2 + C H K 2}$$

$$v 4 = k d p c h k 2 * WIP 1 * \frac{C H K {2}_{p}}{j d p c h k 2 + C H K {2}_{p}}$$

$$v 5 = k p p 5 {3}_{-} C H K 2 * C H K {2}_{p} * \frac{p 5 {3}_{i}}{j p p 5 {3}_{-} C HK2 + p {53}_{i}}$$

$$v 6 = k p p 5 {3}_{-} A T M * A T {M}_{p} * \frac{p 5 {3}_{i}}{j p p 5 {3}_{-} A T M + p 5 {3}_{i}}$$

$$v 7 = k d p p 53 a * WIP 1 * \frac{p 5 {3}_{s 15}}{j d p p 53 a + p 5 {3}_{s 15}}$$

$$v 8 = k p p 53 a * HIPK 2 * \frac{p 5 {3}_{s 15}}{j p p 53 a + p 5 {3}_{s 15}}$$

$$v 9 = k d p p 53 k * \frac{p 5 {3}_{s 46}}{j d p p 53 k + p 5 {3}_{s 46}}$$

$$v 10 = k p s i a h 1 * A T {M}_{p} * \frac{SIAH 1}{j p s i a h 1 + SIAH 1}$$

$$v 11 = k d p s i a h 1 * \frac{SIAH {1}_{p}}{j d p s i a h 1 + {S IAH 1}_{p}}$$

v12 to 20 are reaction rates which relate to gene-expression: mRNA synthesis

$$v 12 = k s p 530 \\ v 13 = k s m d m 20 \\ v 14 = k s m d m 21 * \frac{p 5 {3}_{alg}^{hcmdm 21}}{j s m d m 2 {1}^{hcmdm 21} + p 5 {3}_{alg}^{hcmdm 21}} , \\ where$$

$$p {53}_{alg} = k a l g * p 5 {3}_{i} + p 5 {3}_{S 15} + p 5 {3}_{S 46}$$

$$v 15 = kswip 10$$

$$v 16 = kswip 11 * \frac{p 5 {3}_{s 15}^{h c w i p 11}}{j s w i p 1 {1}^{h c w i p 11} + p 5 {3}_{s 15}^{h c w i p 11}}$$

$$v 17 = k s p 210$$

$$v 18 = k s p 211 * \frac{p 5 {3}_{s 15}^{h c p 211}}{j s p 21 {1}^{h c p 211} + p 5 {3}_{s 15}^{h c p 211}}$$

$$v 19 = k s b a x 0$$

$$v 20 = k s b a x 1 * \frac{p 5 {3}_{s 46}^{h c b a x 1}}{j s b a x {1}^{h c b a x 1} + p 5 {3}_{s 46}^{h c b a x 1}}$$

v21 to v24 are reaction rates which relate to translation: protein synthesis
v21=kpsp53*p53m
v22=kshipk2
v23=kpswip1*WIP1m
v24=kpsmdm2*MDM2m

And finally v25 to v36 are reaction rates which relate to degradation of mRNAs and proteins

$$v 25 = k d p 53 * p 5 {3}_{i} * ( \frac{M D M {2}^{hcdeg}}{M D M {2}^{hcdeg} + j u p 5 {3}^{hcdeg}} ) \\ v 26 = k d p 53 a * p 5 {3}_{s 15} * ( \frac{M D M {2}^{hcdeg}}{M D M {2}^{hcdeg} + j u p 53 {s}^{hcdeg}} ) \\ v 27 = k d p 53 k * p 5 {3}_{s 46} * ( \frac{M D M {2}^{hcdeg}}{M D M {2}^{hcdeg} + j u p 53 {s}^{hcdeg}} ) \\ v 28 = k d h i p k 2 * HIPK 2$$

$$v 29 = kd p 53 m * p 53 m$$

$$v 30 = k d w i p 1 m * WIP 1 m$$

$$v 31 = kd w i p 1 * WIP 1$$

$$v 32 = kdmdm 2 m * MDM 2 m$$

$$v 33 = k d m d m 2 * M D M 2$$

$$v 34 = k d p 21 m * p 21 m$$

$$v 35 = kdbaxm * BAXm$$

$$v36 = k u h i p k 2 * SIAH 1 * \frac{HIPK 2}{j u h i p k 2 + HIPK 2}$$

The rate equations above comprise several constant parameters (e.g. j, k and h), at least some of which have to be estimated using input data. There are two main groups of parameters: those which are patient specific and those which are global parameters applicable to all patients. The general notation for the parameters is that k are rate constants, j are Michaelis or Hill constants and n are the Hill exponents, often also called Hill coefficients. The k constants further use the following standard notation: kp for phosphorylation, kdp for dephosphorylation, ksp for mRNA synthesis, kps for protein synthesis, kd for degradation followed by the gene/protein name, e.g. “atm”. In other words, kpatm is the rate constant for ATM phosphorylation. The definitions for each parameter are set out below.

| Patient- | |
|---|---|
| specific | |
| parameter | Definition |
| ATM_tot | Protein expression of ATM. ATM_tot = ATM + ATMp |
| CHK2_tot | Protein expression of CHK2. CHK2_tot = CHK2 + CHK2p |
| SIAH1_tot | Protein expression of SIAH1/WSB1. SIAH1_tot = |
| | SIAH1 + SIAH1p |
| ksp530 | Basal synthesis parameter for p53 mRNA |
| ksmdm20 | Basal synthesis parameter for MDM2 mRNA |
| kswip10 | Basal synthesis parameter for WIP1 |
| kshipk2 | Rate parameter for HIPK2 translation (protein synthesis |
| | from mRNA) |

| Global | |
|---|---|
| parameters | Definition |
| kpatm | Rate constant for ATM phosphorylation |
| kdpatm | Rate constant for ATMp dephosphorylation |
| jpatm | Michaelis constant (often called K_{M}) for ATM |
| | phosphorylation |
| jdpatm | Michaelis constant for ATMp dephosphorylation |
| kpchk2 | Rate constant for CHK2 phosphorylation |
| kdpchk2 | Rate constant for CHK2 dephosphorylation |
| jpchk2 | Michaelis constant for CHK2 phosphorylation |
| jdpchk2 | Michaelis constant for CHK2 dephosphorylation |
| kpp53_ATM | Rate constant for p53 phosphorylation at S15 by ATMp |
| jpp53_ATM | Michaelis constant for phosphorylation at S15 by ATMp |
| kpp53_CHK2 | Rate constant for p53 phosphorylation at S15 by CHK2p |
| jpp53_CHK2 | Michaelis constant for p53 phosphorylation at S15 by |
| | CHK2p |
| kdpp53a | Rate constant for p53s15 dephosphorylation |
| jdpp53a | Michaelis constant for p53s15 dephosphorylation |
| kpp53a | Rate constant for p53 phosphorylation at S46 |
| jpp53a | Michaelis constant for p53 phosphorylation at S46 |
| kdpp53a | Rate constant for p53s46 dephosphorylation |
| jdpp53a | Michaelis constant for p53s46 dephosphorylation |
| kpsiah1 | Rate constant for SIAH1/WSB1 phosphorylation |
| jpsiah1 | Michaelis constant for SIAH1/WSB1 phosphorylation |
| kdsiah1 | Rate constant for SIAH1/WSB1 dephosphorylation |
| jdsiah1 | Michaelis constant for SIAH1/WSB1 dephosphorylation |
| ksmdmd21 | Rate constant for p53-induced MDM2 mRNA synthesis |
| jsmdmd21 | Michaelis constant for p53-induced MDM2 mRNA |
| | synthesis |
| hcmdm21 | Hill coefficient for p53-induced MDM2 mRNA |
| | synthesis |
| kalg | Factor describing transcriptional activity of |
| | unphosphorylated p53 relative to phosphorylated |
| | p53 for inducing MDM2 mRNA synthesis |
| kswip11 | Rate constant for p53-induced WIP1 mRNA synthesis |
| jswip11 | Michaelis constant for p53-induced WIP1 mRNA |
| | synthesis |
| hcwip11 | Hill coefficient for p53-induced WIP1 mRNA synthesis |
| ksp210 | Rate constant for basal p21 mRNA synthesis |
| ksp211 | Rate constant for p53-induced p21 mRNA synthesis |
| jsp111 | Michaelis constant for p53-induced p21 mRNA |
| | synthesis |
| hcp211 | Hill coefficient for p53-induced p21 mRNA synthesis |
| ksbax0 | Rate constant for basal BAX mRNA synthesis |
| ksbax1 | Rate constant for p53-induced BAX mRNA synthesis |
| jsbax1 | Michaelis constant for p53-induced BAX mRNA |
| | synthesis |
| hcbax1 | Hill coefficient for p53-induced BAX mRNA synthesis |
| kpsp53 | Rate constant for p53 protein synthesis |
| kshipk2 | Rate constant for HIPK2 protein synthesis |
| kpswip1 | Rate constant for WIP1 protein synthesis |
| kpsmdmd2 | Rate constant for MDM2 protein synthesis |
| kdp53 | Rate constant for unphosphorylated p53 protein |
| | degradation |
| jup53 | Hill constant for unphosphorylated p53 protein |
| | degradation |
| hcdeg | Hill coefficient for unphosphorylated p53 protein |
| | degradation |
| kdp53a | Rate constant for p53s15 protein degradation |
| jup53s | Hill constant for p53s15 and p53s46 protein degradation |
| hcdeg | Hill coefficient for p53s15 and p53s46 protein |
| | degradation |
| kdp53k | Rate constant for p53s46 protein degradation |
| kdhipk2 | Rate constant for HIPK2 protein degradation |
| kdp53m | Rate constant for p53 mRNA degradation |
| kdwip1m | Rate constant for WIP1 mRNA degradation |
| kdwip1 | Rate constant for WIP1 protein degradation |
| kdmdm2m | Rate constant for MDM2 mRNA degradation |
| kdmdm2 | Rate constant for MDM2 protein degradation |
| juhipk2 | Hill constant for HIPK2 protein degradation |
| kdp21 | Rate constant for p21 mRNA degradation |
| kdbax | Rate constant for BAX mRNA degradation |
| DDR | Amount of DNA damage, dosage of DNA damaging drug |

The initial conditions for each state are shown in the table below and as shown the states for which there are patient specific parameters (as explained in more detail below), there is not a fixed numeric value (e.g. 0) but a parameter value for the patient:

| ATM | ATM_p | p53m | p53_i | p53s15 | p53s46 |
|---|---|---|---|---|---|
| ATM_tot | 0 | 0.717 | 0.079 | 0 | 0 |

| SIAH1 | SIAH1p | HIPK2 | WIP1m | CHK2 | CHK2p |
|---|---|---|---|---|---|
| SIAH1_tot | 0 | 0.018 | 0.127 | CHK2_tot | 0 |

| MDM2m | MDM2 | p21m | BAXm |
|---|---|---|---|
| 0.003 | 0.190 | 0.012 | 0.160 |

The parameters in the model were estimated using a global parameter optimization method such as GLSDC implemented in the PEPSSI software as described for example in “Performance of objective functions and optimisation procedures for parameter estimation in system biology models” by Degasperi et al published in NPJ Syst Biol Appl 3, 20 (2017). To assess the uncertainty of these estimates and the associated predictions, a Monte-Carlo based approach that randomly changes the initial parameter guesses 96 times and re-fits the model systematically evaluating the probability of these parameters to fit the experimental data was used. This method provides a large set of good-fitting parameter estimates and an estimate for how likely a correct solution is found by chance rather than by identifying the correct experimental parameter values. Merely as examples the table below shows three sets of these estimates for all the parameters which are not affected by the type of treatment.

| kpchk2 | jpchk2 | kdpchk2 | jdpchk2 | kdpp53k | jdpp53k |
|---|---|---|---|---|---|
| 39.95245 | 0.317749 | 20.63249 | 0.999747 | 3.581373 | 0.126013 |
| 39.94037 | 0.352166 | 20.40821 | 0.999747 | 3.677789 | 0.124187 |
| 39.98596 | 0.396237 | 20.70913 | 0.999843 | 2.026363 | 0.045067 |
| kpp53_ATM | kpp53_CHK2 | jpp53_ATM | jpp53_CHK2 | ksmdm21 | jsmdm21 |
| 1.98137 | 31.29065 | 0.012328 | 0.561453 | 0.795847 | 0.32057 |
| 1.581683 | 31.4206 | 0.010013 | 0.520408 | 0.802444 | 0.328572 |
| 19.41021 | 6.669001 | 0.010016 | 0.414508 | 0.757492 | 0.298957 |
| kdpp53a | jdpp53a | kpp53a | jpp53a | jup53 | jup53s |
| 39.99165 | 0.253686 | 39.83628 | 0.01 | 0.004473 | 9.98853 |
| 39.98745 | 0.261434 | 39.98183 | 0.010008 | 0.003626 | 9.999533 |
| 39.97056 | 0.173038 | 30.9098 | 0.010003 | 0.002743 | 0.100744 |
| kpsiah1 | jpsiah1 | kdpsiah1 | jkpsiah1 | sf_s15 | sf_s46 |
| 1.958091 | 0.045366 | 1.622702 | 0.045967 | 1.152567 | 1.047521 |
| 2.023932 | 0.025361 | 1.868788 | 0.07929 | 1.167204 | 1.070399 |
| 3.761721 | 0.014066 | 6.594117 | 0.815415 | 1.070264 | 1.660236 |
| kswip11 | jswip11 | kdp53 | kdp53a | juhipk2 | |
| 0.861278 | 0.499969 | 7.999482 | 0.100019 | 0.001009 | |
| 0.853901 | 0.499976 | 7.99864 | 0.100097 | 0.001005 | |
| 0.851104 | 0.499961 | 7.091471 | 0.100004 | 0.014242 | |

As shown above, many of the constants have similar values for each estimate. However, for example, for kdpp53k and jdpp53k, the third value is significantly different to each of the other two values for these parameters. This may be explained because there is a complex relationship between at least some of the parameters and thus some of the parameters are highly correlated with each other. Regardless of these variations in parameters, the model is reliable and consistent in its predictive results.

There are also a few constants whose values are dependent on the nature of the treatment and three example sets of values are set out below.

Results fitted to doxorubicin treatments

| Parameter | kpatm | jpatm | kdpatm | jdpatm |
|---|---|---|---|---|
| Estimate #1 | 1.746184 | 0.999825 | 0.100007 | 0.99994 |
| Estimate #2 | 1.764465 | 0.999964 | 0.100011 | 0.999993 |
| Estimate #3 | 1.444259 | 0.999832 | 0.100032 | 0.999806 |

Results fitted to radiation treatments

| Parameter | kpatm | jpatm | kdpatm | jdpatm |
|---|---|---|---|---|
| Estimate #1 | 0.683308 | 0.010004 | 4.619372 | 0.040022 |
| Estimate #2 | 0.692243 | 0.010004 | 4.654454 | 0.039543 |
| Estimate #3 | 0.646103 | 0.010002 | 4.043788 | 0.037907 |

There are also constants that do not have to be estimated. For parameter estimation, the total protein concentrations for ATM, CHK, and SIAH1 were normalised resulting in the following values for these constants. These values would be used as the initial states as shown above.

| Parameter | ATM_tot | SIAH1_tot | CHK2_tot |
|---|---|---|---|
| Value | 1 | 1 | 1 |

Thus as shown above, kpatm has a value in the range of approximately 1.44 to 1.76, jpatm and jdaptm have an approximate value of 1, kdpatm has an approximate value of 0.1 when fitting to doxorubin treatment. Similarly, kpatm has a value in the range of approximately 0.65 to 0.69, jpatm has an approximate value of 0.01, kdpatm has a value in the range of approximately 4.04 to 4.65 and jdpatm has a value in the range of approximately 0.038 to 0.04 when fitting to radiation treatment.

It is also noted that because the p53s15 and p53s46 measurements were in arbitrary units, two scaling factors were estimated to scale the measurement data to the normalised model units: yₚ₅₃ₛ₁₅=sf_s15*(p53s15+p53s46), yₚ₅₃ₛ₄₆=sf_s46*p53s46, where yₚ₅₃ₛ₁₅, yₚ₅₃ₛ₄₆ denote the protein measurements; sf_s15, sf_s46 the scaling factors; and p53s15, p53s46 the model states for p53s15 and p53s46, respectively. Here, the sum of the p53s15 and p53s46 model states was used to fit the yₚ₅₃ₛ₁₅ data, because the corresponding antibody used for this measurement detected both phosphorylated p53 forms.

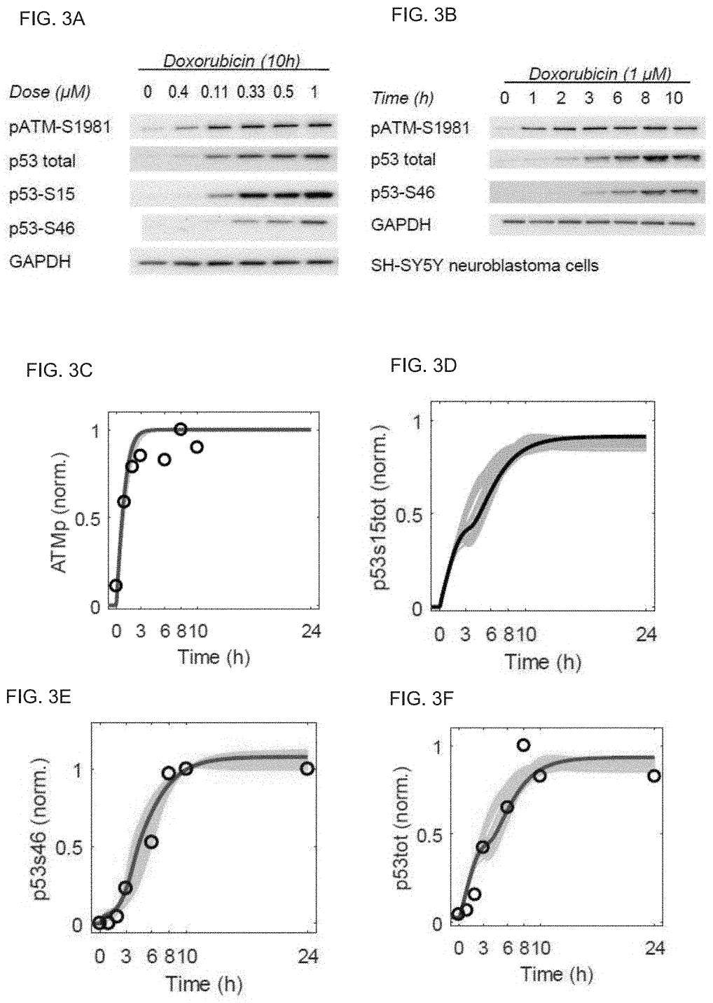

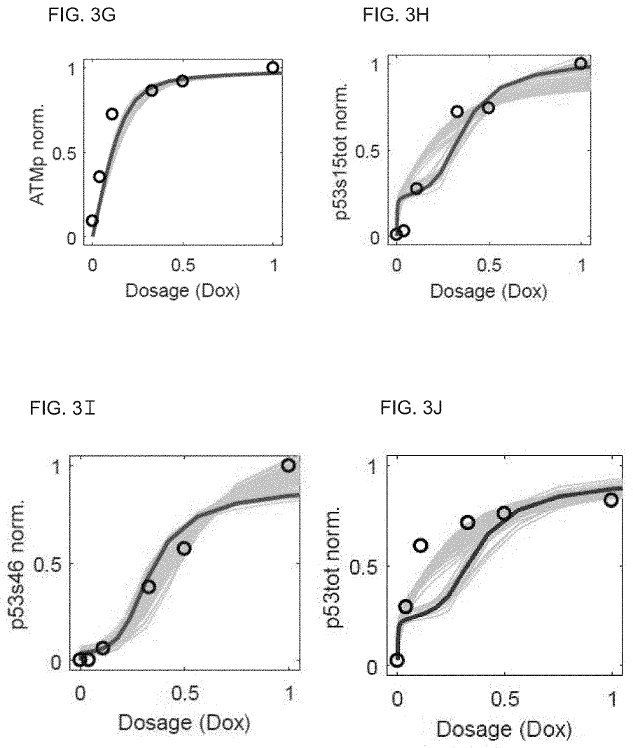

FIGS. 3A to 3J show the results from a first calibration of the model to ensure that it is consistent with DDR dynamics observed in p53-wild-type neuroblastoma cells from our own experiments. FIGS. 3A and 3B show timecourse and dose-response data describing the activity of most model components in SH-SY5Y neuroblastoma cells which was generated by the applicants from SH-SY5Y neuroblastoma cells. FIG. 3A shows representative Western blots the model components in a 10 hour window treated with different doses of doxorubicin in SH-SY5Y neuroblastoma cells and FIG. 3B shows representative Western blots for the markers varying with time for a fixed dose of doxorubicin. It will be appreciated that doxorubicin is just one suitable chemotherapeutical agents, other examples include cisplatin, oxaliplatin, methotrexate and daunorubicin.

The data generated in FIGS. 3A and 3B was then used to generate FIGS. 3C to 3F which plot the normalised variation against time in both the measured and modelled data for the markers ATMp, p53s15tot, p53s46 and p53tot and FIGS. 3G to 3J which plot the normalised variation against dosage. The simulation results show that our model predicted the measured dynamics well including the p53pS15 and p53pS46 states. This indicates that the model contains all components and topological features necessary to faithfully predict differential p53 regulation in DDR conditions. The predicted shapes of the time and dose responses matched those measured. Crucially, the qualitative dynamics and order of events matched for all model components. As predicted by the model, the neuroblastoma cells activated ATM first and at low dosages, followed by transitions from p53^{pS15} to p53^{pS46} later and at higher dosages.

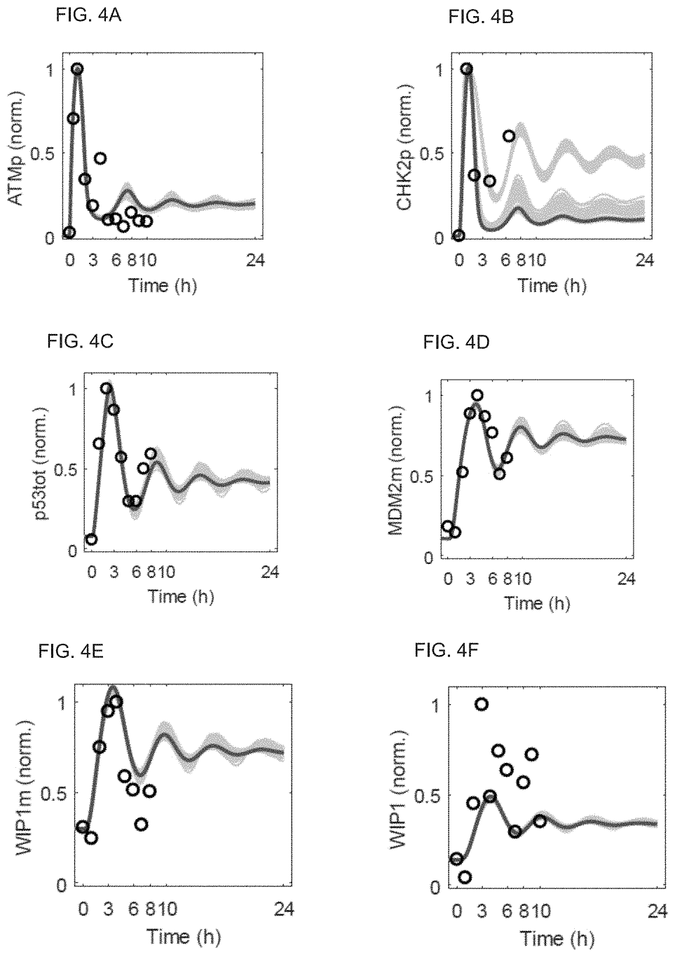

FIGS. 4A to 4F show the results from a second calibration of the model to ensure that it is also consistent with experimental data from the literature. These figures are plotted using experimentally measured time-course data from MCF7 breast-cancer cells from the literature, for example “p53 dynamics control cell fate” by Purvis, J. E., et al published in Science 336, 1440-1444 (2012) or “Stimulus-dependent dynamics of p53 in single cells” by Batchelor et al published in Molecular systems biology 7, 488 (2011). In line with the results shown in FIGS. 3A to 3J, the predicted shapes of the time and dose responses matched those measured.

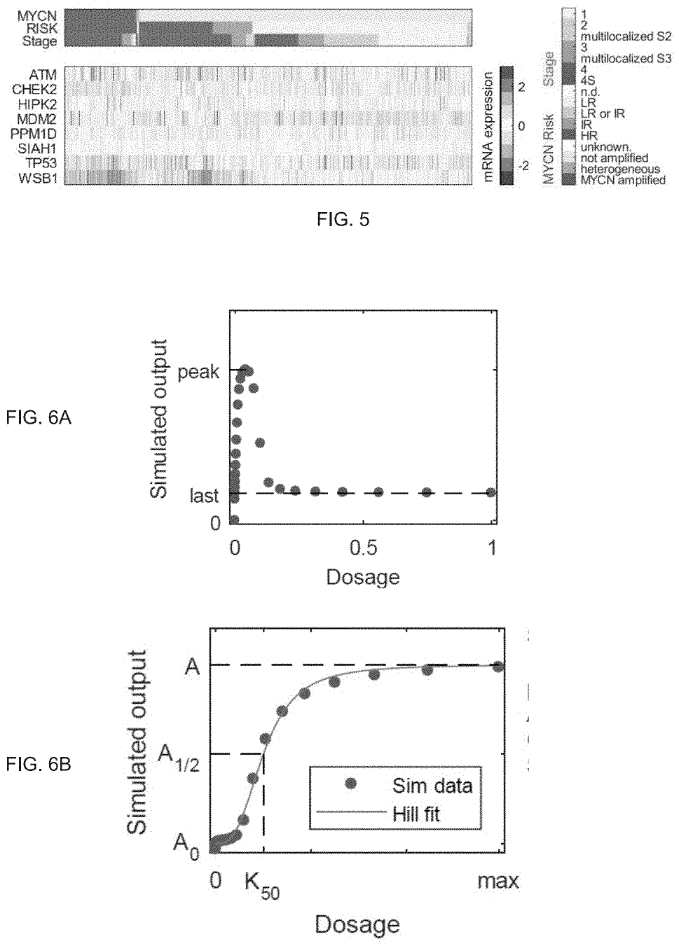

FIGS. 5 to 8B illustrate how the generic p53 model above can be personalised to generate patient-specific simulations which can be used to gain insight into individual drug-response and resistance mechanisms. The model is personalised by incorporating gene-expression tumour data from patients as shown in FIG. 5. As set out above, the model contains seven patient-specific parameters and it is these parameters which are estimated from tumour data: ATMtot, CHK2tot, ksp530, ksmdm20, kswip10, SIAH1tot, kshipk2.

As a technical requirement, the approach assumes that tumour signalling data reflect measurements of steady-state DDRs, which is reasonable considering that changes in gene-expression caused by natural tumour evolution occur on much slower timescales than acute treatment-induced signalling changes. The approach is based on the principle that individual differences in the signalling behaviour emerge from gene-expression differences. For each gene, a basal gene-expression parameter is presumed to be patient-specific. It was demonstrated that these patient-specific basal expression rates can be estimated from the tumour data by matching the model-predicted and measured gene expression values at steady state.

It is noted that a unique solution to the problem of estimating the patient specific parameters might not exist. In the paper “On the personalised modelling of cancer signalling” by Fey et al published in IFAC-PapersOnLine 49, 312-317 (2016), general constraints on the equations of the model that have to be fulfilled in order for such a unique solution to exist have been formulated. As set out below, it is shown that these conditions are fulfilled for the p53 model, and compute an explicit formula for estimating the patient-specific parameters.

To apply the approach to the p53 model, it is postulated that the tumour data was measured in the absence of DNA-damaging drugs. This is clearly the case in primary samples from untreated patients, and also a fair assumption for relapsed samples that were collected several days or sometimes weeks after the last treatment. The advantage of assuming drug-absence is that the model structure becomes very simple, because most model components reside in their inactive steady states. As a result, most model equations are decoupled, which facilitates calculating the solution. Two scenarios can be distinguished:

In the first scenario, there are model components that are completely decoupled (e.g. ATM, CHK2, SIAH1) and are characterised by a conserved moiety. In such a scenario, model-personalisation is simple. As explained in “Patient-specific simulations of JNK activity predict the survival of neuroblastoma patients” by Fey et al published in Sci Signal 8, ra130 (2015), the total protein concentration of each of these components is a time-invariant parameter in the model that can easily be adjusted according to the measurement. For example, let CHK2 be 30% upregulated in a patient, then the concentration of total CHK2 for this patient is adjusted to 1.3 times the nominal value. Here, the nominal value refers to the parameter value from model calibration.

In the second scenario, the other model components are still coupled through gene-regulatory interactions and thus indirect estimations are required. The most complicated subsystems in our model are the p53 and MDM2 module involving a transcriptional feedback loop as described in detail above in relation to FIG. 2. In the following, it is shown how for different patients, their basal gene expression rates of p53 and MDM2 can be obtained from their tumour data, which illustrates the general procedure.

As mentioned, zero-input, steady-state measurements are assumed for the tumour data. For zero input, all phosphorylation reactions in the model become zero over time, all of p53 is in its unphosphorylated form, and the steady state equations for p53 and MDM2 are:

$$\begin{matrix} p 53 : \\ 0 = ksp 530 - kd p 53 m * p 53 m , \\ 0 = k p s p 53 * p 53 m - k d p 53 * p 5 {3}_{i} * ( \frac{M D M {2}^{h c d e g}}{M D M {2}^{h cdeg} + jup {53}^{h c d e g}} ) , & \\ MDM 2 : & \\ 0 = ks m d m 20 + k s m d m 21 * \frac{p 5 {3}_{i}^{h c m d m 21}}{jsmd 2 m {21}^{h c m d m 21} + p 5 {3}_{i}^{h c m d m 21}} - k d m d m 2 m * M D M 2 m , \\ 0 = kpsmdm 2 * MD M 2 m - k d m d m 2 * M D M2 , & \end{matrix}$$

These equations estimate the patient-specific parameters ksp530 and ksmdm20 based on p53m and MDM2m, the measured p53 and MDM2 gene expression data from a patient tumour sample. Solving for the patient specific parameters ksp530 and ksmdm20 yields

$$ksp 530 = kdp 53 m * p53 m , \\ ksmdm 20 = kdmdm 2 m * MD M 2 m - k s m d m 21 * \frac{p 5 {3}_{i}^{h c m d m 21}}{jsmd 2 m {21}^{h c m d m 21} + p 5 {3}_{i}^{h c m d m 21}} , \\ with$$

$$p {53}_{i} = \frac{k p s p 53 * p 53 m}{kd p 5 \; 3} \frac{M D M {2}^{h c d e g} + jup 5 {3}^{h c d e g}}{M D M {2}^{h c d e g}} , \\ and$$

$$MDM 2 m = MDM 2 m * \frac{kpsmd 2 m 2}{kd 2 md 2 m 2} ,$$

where:

ksp530 and ksmdm20 are the estimates of the patient-specific parameters, and denote the basal mRNA synthesis rates of p53 and MDM2, respectively;

p53m and MDM2m denote the measured mRNA concentrations of p53 and MDM2;

p53ᵢ, MDM2 are the calculated protein concentrations of p53 and MDM2;

kdp53m, kdmdm2m, ksmdm21, jsmdm21, hcmdm21, kpsp53, kdp53, jup53, hcdeg, kpsmdm2, kdmdm2 are known parameters as defined in the table above.

The complete set of formulae for all the patient specific parameters for all the components (genes) in the model is summarised below. In the table below, x denotes the measured mRNA data on a log 2 scale with the subscript indicating the corresponding gene.

| Model | Formula for | mRNA data to use |
|---|---|---|
| component | patient-specific parameter | (gene name/symbol) |
| ATM | ATM_{tot,patient} = 2^{x}^{_{ATM}} * ATMₜₒₜ | ATM and/or ATR |
| CHK2 | CHK2_{tot,patient} = 2^{x}^{_{CHEK2}} * CHK2ₜₒₜ | CHEK2 and/or CHEK1 |
| p53 | ksp530 = kdp53m * 2^{x}^{_{TP53}} | TP53 and/or TP63, TP73 |
| MDM2 | $\begin{matrix}ksmdm 20 = kdmdm 2 m * {2}^{{x}_{MDM 2}} - \\ ksmdm 21 * \frac{p {53}_{i}^{hcmdm 21}}{jsmdm {21}^{hcmdm 21} + p {53}_{i}^{hcmdm 21}} ,\end{matrix}$ | MDM2 and/or MDM4, MDMX |
| | where: | |
| | $\begin{matrix}p {53}_{i} = \frac{kpsp 53 * p 53 m}{kdp 53} \frac{MDM {2}^{hcdeg} + jup {53}^{hcdeg}}{MDM {2}^{hcdeg}} , \\ MDM 2 = {2}^{{x}_{MDM 2}} * \frac{kpsmdm 2}{kdmdm 2} .\end{matrix}$ | |
| WIP1 | kswip10 = kdwip1m * 2^{x}^{_{WIP1}} | PPM1D |
| SIAH1 | SIAH1_{tot,patient} = 2^{(x}^{_{SIAH1}}^{+x}^{_{WSB1}}⁾ * SIAH1ₜₒₜ | SIAH1 and/or |
| | | WSB1, SIAH2 |
| HIPK2 | kshipk2 = 2^{x}^{_{HIPK2}} * pkshipk2 | HIPK2 and/or HIPK1 |

Similarly, the patient specific parameters can also be estimated from protein expression measurements from a patient tumour sample. Solving for the patient specific parameters ksp530 and ksmdm20 yields:

$$ksp 530 = kdp 53 m * p5 \; 3 m$$

$$with$$

$$p 5 \; 3 m = \frac{k d p 53}{k p s p 53} * p 53 i * \frac{M D M {2}^{h c d e g}}{M D M {2}^{h c d e g} + j u p 5 {3}^{h c d e g}} , \\ ksmdm 20 = kdmdm 2 m * MD M 2 m - k s m d m 21 * \frac{p 53 {i}^{h} c m d m 21}{p 53 {i}^{h} c m d m 21 + j s m d m 22 {1}^{h} c m d m 21}$$

$$with$$

$$MDM 2 m = \frac{k d m d m 2}{k p s m d m 2} * M D M 2 ,$$

where p53i and MDM2 denote the measured protein expression values for p53 and MDM2, respectively. The following table show the complete set of equations for solving the patient-specific parameters from protein expression data for all proteins in the model.

In the table below, y denotes the measured protein data on log 2 scale with the subscript indicating the corresponding protein.

| Model | Formula for | Protein data to use |
|---|---|---|
| component | patient-specific parameter | (protein name) |
| ATM | ATM_{tot,patient} = 2^{y}^{_{ATM}} * ATMₜₒₜ | ATM and/or ATR |
| CHK2 | CHK2_{tot,patient} = 2^{y}^{_{CHK2}} * CHK2ₜₒₜ | CHK2 and/or CHK1 |
| p53 | ksp530 = kdp53m * p53m | p53 and/or p63, p73 |
| | where: | |
| | $p 53 m = \frac{kdp 53}{kpsp 53} * p 53 i * \frac{MDM {2}^{hcdeg}}{MDM {2}^{hcdeg} + jup {53}^{hcdeg}}$ | |
| MDM2 | $\begin{matrix}ksmdm 20 = kdmdm 2 m * MDM 2 m - \\ ksmdm 21 \frac{{( {2}^{{y}_{p 53}} )}^{hcmdm 21}}{{( {2}^{{y}_{p 53}} )}^{hcmdm 21} + jsmdm {221}^{hcmdm 21}}\end{matrix}$ | MDM2 and/or MDM4, MDMX |
| | where: | |
| | $MDM 2 m = \frac{kdmdm 2}{kpsmdm 2} * {2}^{{y}_{MDM 2}}$ | |
| WIP1 | kswip10 = kdwip1m * 2^{y}^{_{WIP1}} | WIP1 |
| SIAH1 | SIAH1_{tot,patient} = 2^{(y}^{_{SIAH1}}^{+y}^{_{WSB1}}⁾ * SIAH1ₜₒₜ | SIAH1 and/or |
| | | WSB1, SIAH2 |
| HIPK2 | kshipk2 = 2^{y}^{_{HIPK2}} * pkshipk2 | HIPK2 and/or HIPK1 |

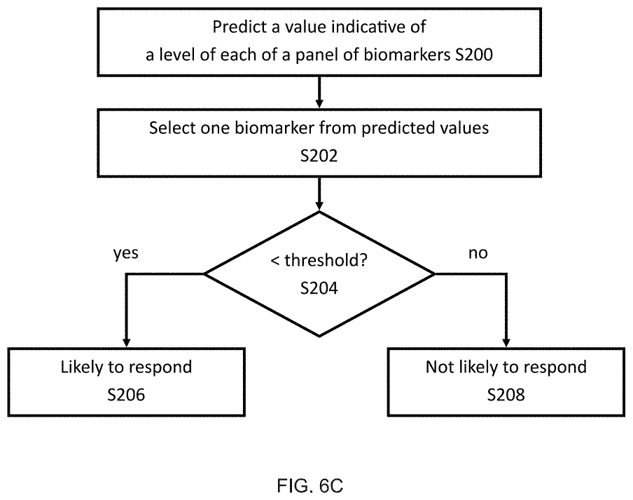

To gain a more comprehensive picture of the model's prognostic utility, features including peak values (peak), half-activation constants (K50), amplitude (A) and Hill-exponent (n) from all model components were tested 1-by-1. FIGS. 6A and 6B illustrate some of these model outputs as functions of the input u where u denotes the dosage. For example, FIG. 6A shows the peaked outputs from p53s15 which is the numerical output from the simulation, e.g. p53s15_peak=max (p53s15(u)). FIG. 6B shows the amplitude outputs for p53s46. The half-activation constant p53s46_K50 is the value for u for which p53s46(u)=½ max(p53s46). This can be obtained by fitting a Hill function, e.g. xⁿ/(xⁿ+K50ⁿ) to the simulated dose response.

FIG. 6A illustrates how the model above may be used. The model is used to predict a value which is indicative of the level of each of the biomarkers (step S200). These features from the models can then be used in a Kaplan Meier survival analysis for example as described in the paper “Signaling pathway models as biomarkers: Patient-specific simulations of JNK activity predict the survival of neuroblastoma patients” by Fey et al. Sci Signal 8, ra130 (2015) to stratify the patients. In line with known analysis techniques as described for example in the Fey 2015 paper, Cox regression can be used to quantify the difference between the stratified groups in form of a hazard ratio and a logrank test can be used to evaluate statistical significance. Hazard ratios >1 indicate an effect, p-values <0.05 and <0.01 indicate statistical significance at the 95% and 99% confidence level.

The stratification step can be used to stratify the whole patient cohort or to stratify patients who have already been grouped into high, low and/or intermediate risk groups, for example using the current Childrens Oncology Group (COG) risk classification, e.g. as described in “Neuroblastoma” by Maris et al. published in Lancet 369(9579):2106-20 (2007). The stratification step applied in the present application stratifies the cohorts into patients having a favourable and unfavourable prognosis to treatment by chemotherapy. The stratification may be done by deriving a threshold for each marker as described below, e.g. a p53s15peak threshold for the output shown in FIG. 6A. One of the markers may then be selected (step S202) and compared to a threshold (step S204). All patients having an output value for that marker below the threshold are classified as having a favourable prognosis (step S206) and all patients having an output value equal to or above the threshold value are classified as having an unfavourable prognosis (step S208). The patient survival of the two groups is then plotted over time. Such a classification or stratification of patients, allows the treatment for a patient within each risk group to be tailored to that individual patient rather than all patients being treated in the same way.

The threshold may be derived by know techniques, for example Kaplan Meier scanning as described for example in the Fey 2015 paper. Kaplan Meier scanning works as follows:
1. Place the patient with the lowest marker value into the low group and all other patients into the high group.
2. Assess statistical significance of the group difference in survival using the logrank test.
3. Add the patient with the next highest maker value into the low group.
4. Iterate from point 2 until all but one patients are in the low group.
5. The iteration with the lowest logrank p-value defines the optimal threshold.
   It will be appreciated that this is just one illustrative method for calculating the threshold and other suitable methods may be used.

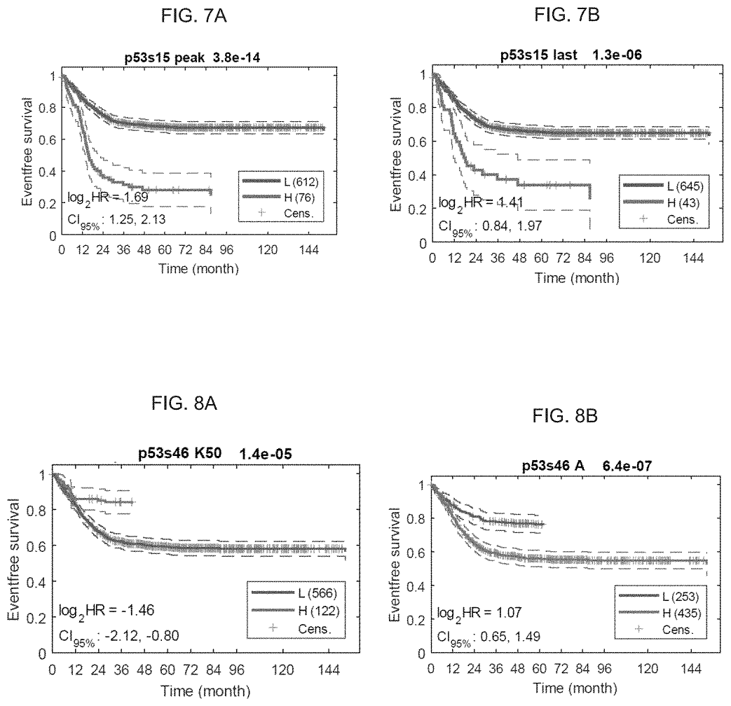

For example, FIG. 7A stratifies the group of 688 patients into 612 patients with favourable prognosis and 76 patients with unfavourable prognosis based on their individual peak value for p53s15 using a p53s15_peak threshold. Similarly, FIG. 7B stratifies the group of 688 patients into 645 patients with a favourable prognosis and 43 patients with an unfavourable prognosis based on their individual last value for p53s15 when compared to a p53s15_last threshold. FIG. 8A stratifies the group of 688 patients into 566 patients having a favourable prognosis and 122 patients having an unfavourable prognosis based on their individual half activation threshold for p53s46 when compared to a p53s46_K50_threshold. FIG. 8B stratifies the group of 688 patients into 253 patients having a favourable prognosis and 435 patients having an unfavourable prognosis using the amplitude value for p53s46 and comparing it to a p53s46_amplitude threshold.

As can be seen from the hazard ratios and confidence interval which are shown on each of FIGS. 7A to 8B, the peak value of the simulated p53s15 peak output had the most prognostic value for chemotherapy response. The following table summarises the results for all the markers and based on the Hazard ratio and the p-value, in addition to p53s15_peak, CHK2p_peak shows excellent prognostic value, as indicated by a logrank test p-value of 2.2*10⁻²¹ and a hazard ratio of 3.4.

| Predictor / | | p-value | Hazard | HR 95% | HR 95% |
|---|---|---|---|---|---|
| marker | Threshold | (logrank) | ratio | CI lower | CI upper |
| CHK2p_peak | 1.15468 | 2.2E−21 | 3.400 | 2.641 | 4.377 |
| p53s15_peak | 0.24654 | 3.75E−14 | 3.231 | 2.38 | 4.378 |
| MDM2_K50 | 0.00877 | 4.42E−13 | 0.387 | 0.300 | 0.501 |
| WIP1_A1 | 0.09958 | 1.99E−11 | 4.120 | 2.724 | 6.232 |
| MDM2_A1 | 3.59510 | 4.25E−11 | 4.128 | 2.708 | 6.291 |
| HIPK2_K50 | 0.20867 | 7.07E−09 | 0.446 | 0.339 | 0.586 |
| p53s46_A1 | 0.12542 | 6.4E−07 | 2.093 | 1.565 | 2.800 |
| p53s46_peak | 1.06918 | 8.72E−07 | 2.024 | 1.528 | 2.681 |
| WIP1_K50 | 0.01592 | 3.2E−06 | 0.547 | 0.425 | 0.705 |
| p53s46_K50 | 0.38006 | 1.44E−05 | 0.363 | 0.230 | 0.574 |
| MDM2_n | 1.00000 | 0.000237 | 0.551 | 0.401 | 0.757 |
| SIAH1p_peak | 0.756191 | 0.000877 | 0.400297 | 0.233436 | 0.686431 |
| HIPK2_A1 | 0.081506 | 0.005843 | 0.371324 | 0.183579 | 0.751074 |
| ATMp_peak | 0.85392 | 0.007276 | 1.409811 | 1.097065 | 1.811713 |
| WIP1_n | 2.007336 | 0.010693 | 0.516974 | 0.311497 | 0.857992 |
| HIPK2_n | 1.66013 | 0.024691 | 1.334296 | 1.03743 | 1.71611 |
| p53s46_n | 2.089183 | 0.170409 | 1.218777 | 0.918493 | 1.617231 |

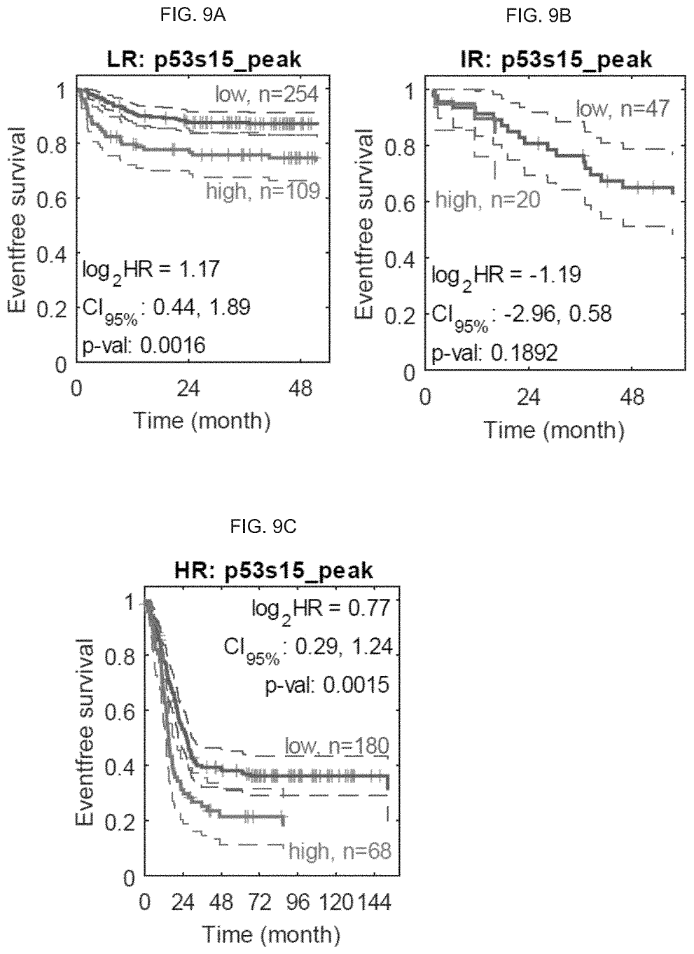

In light of the promising prognosis shown in FIG. 7A, FIGS. 9A to 9C show an alternative method for stratification of patients using the p53s15 peak value. In this case, rather than stratifying the whole cohort, the low, intermediate and high risk groups which are generated by the Children's Oncology Group (COG) are stratified to provide a fine grained stratification. FIG. 9A shows the stratification of 363 low risk patients into 254 with a favourable prognosis and 109 with an unfavourable prognosis. FIG. 9B shows the stratification of 67 intermediate risk patients into 47 with a favourable prognosis and 20 with an unfavourable prognosis. FIG. 9C shows the stratification of the remaining 248 high risk patients into 180 with a favourable prognosis and 68 with an unfavourable prognosis.

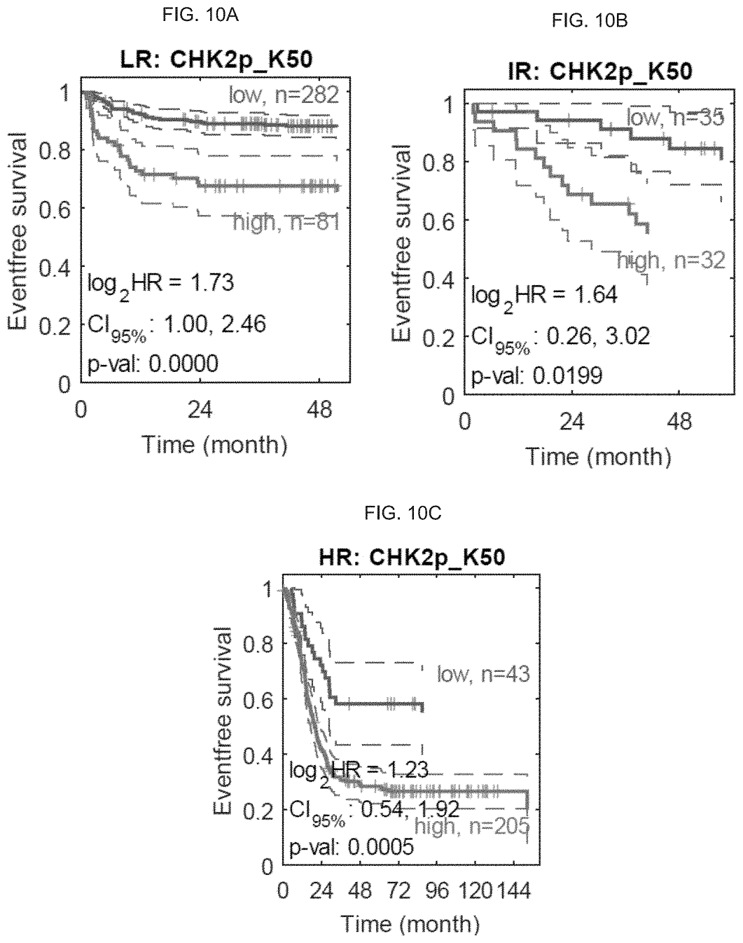

A similar stratification is shown for the CHK2p half-activation threshold (K50). FIG. 10A shows the stratification of 363 low risk patients into 282 with a favourable prognosis and 81 with an unfavourable prognosis. FIG. 10B shows the stratification of 67 intermediate risk patients into 35 with a favourable prognosis and 32 with an unfavourable prognosis. FIG. 10C shows the stratification of the remaining 248 high risk patients into 43 with a favourable prognosis and 205 with an unfavourable prognosis.

In each of FIGS. 9A to 10C, patients of the same risk classification as defined by COG obtain the same, or depending on regional/national differences, very similar treatments. The further stratification into favourable and unfavourable prognosis was obtained by applying Kaplan Meier analysis and Cox regression to the model outputs. It is noted that in each of the graphs, the patient group having a favourable prognosis is labelled L for low risk and the patient group having an unfavourable prognosis is labelled H for high risk. However, the proposed stratification strategy is not the same as the risk stratification used in COG and thus the terms favourable and unfavourable prognosis have been used to distinguish the two types of stratification. It is noted that the results are successful, for example the hazard ratios for the stratification based on CHK2_K50 of the low, intermediate and high risk cohorts into favourable and unfavourable prognosis are 3.32, 3.12 and 2.35 respectively. It is noted that the hazard ratios are shown in the figures in the log 2 scale, for example 2\hat{ }[1.73, 1.64, 1.23] is equivalent to the listed ratios [3.32, 3.12, 2.35]. Similarly, the p-values are <10⁻⁶, 0.02 and 0.0005.

FIGS. 11A to 11C show a set of results showing the robustness and reliability of the model. In each of FIGS. 11A to 11C, a multivariate Cox regression model was generated. Multivariate Cox regression is a technique which allows a combination of several markers and generates an estimate the relative contribution of each marker to the prediction. Using the standard 10×2 fold cross-validation, the input data was randomly split multiple times with part of the input data being used to train the Cox regression model and the other part being used to validate the Cox regression. The hazard ratios comparing the stratification into unfavourable and favourable outcomes is then generated for each one of the random splits of data. FIG. 11A shows the hazard ratios for the stratification of the entire cohort into unfavourable and unfavourable outcomes for different sets of markers using three different approaches—“both”, “data” and “model”. “Data” uses the mRNA measurements for all genes in the model, i.e. for ATM, CHEK2, TP53, MDM2, PPM1D, SIAH1, HIPK2 and WSB1. “Model” uses the simulated model outputs for peak values of the proteins p53s15, ATMp, CHK2p, SIAH1p, p53s46 and the half activation and amplitude values of the proteins HIPK2, WIP1 and MDM2. “Both” uses both simulated model outputs for the listed proteins and measured mRNA data for the listed genes to stratify the cohort.

Similarly, FIG. 11B shows the hazard ratios between the two groups of favourable and unfavourable prognosis which are generated from the high risk cohort as classified by COG. The hazard ratios for each of the 20 cross-validation runs for each of the “both”, “data” and “model” approaches are shown. Similarly, FIG. 11C shows the hazard ratios between the two groups of favourable and unfavourable prognosis which are generated from the low risk cohort as classified by COG.

In each of FIGS. 11A to 11C, the calculated hazard ratios (in particular the median values) are very similar, even though each hazard ratio is generated using a different randomly sampled subset of the patient data. These cross-validations demonstrate the robustness of the predictions with respect to variations in the composition of the patient cohort, and the reliability of the predictions.

As explained in FIGS. 11A to 11C, the “Data” model uses the mRNA measurements, which can be obtained in any known way, for example as described in the article “Revised Risk Estimation and Treatment Stratification of Low- and Intermediate-Risk Neuroblastoma Patients by Integrating Clinical and Molecular Prognostic Markers” by Oberthuer A. et al. in Clinical Cancer Research 21(8), 1904-1915 2015, for a group of genes, i.e. for ATM, CHEK2, TP53, MDM2, PPM1D, SIAH1, HIPK2 and WSB1. Thus, the data may come from a DNA microarray. Alternatively, as another example, clinically used qrt-PCR assays may be used in mRNA sequencing. A general reference for measuring gene expression is “Transcriptional Biomarkers” Methods Vol 59, Issue 1, Pages 1 to 163 & S1 to S28 edited by Michael W Pfaffl.

The measurements are summed in a weighted sum to create a value V which is indicative of eventfree survival. The model may thus be expressed as:

$$V = {w}_{A T M} A T M ( m ) + {w}_{H E K 2} C H E K 2 ( m ) + {w}_{T P 53} T P 53 ( m ) + {w}_{M D M 2} M D M 2 ( m ) + {w}_{P P M 1 d} P P 1 M ( m ) + {w}_{SIAH 1} SIAH 1 ( m ) + {w}_{HIPK 2} HIPK 2 ( m ) + {w}_{W S B 1} W S B 1 ( m )$$

where wᵢ is the weight for each gene and i(m) is the measurement of the gene.

The weights are calculated using training data to create a best fit or optimal value for each weight. For example, a Cox regression analysis may be performed. As examples, two variations of the weights for each gene are shown below:

| Gene | ATM | CHEK2 | HIPK2 | MDM2 | PPM1D | SIAH1 | TP53 | WSB1 |
|---|---|---|---|---|---|---|---|---|
| Weight 1 | 0.024 | 0.496 | 0.278 | 0.143 | 0.069 | −0.644 | 0.183 | −0.591 |
| Weight 2 | 0.035 | 0.119 | 0.149 | 0.197 | −0.363 | −0.345 | 0.165 | −0.295 |

FIGS. 11D and 11E shows the eventfree survival against time plotting using the weighted sums with each of the weights above. In FIG. 11D, the entire cohort is stratified patients into two groups. 378 patients are separated into a group having a favourable prognosis and 310 into a group having a non-favourable prognosis. The relative risk between the two groups as measured by hazard ratio was 5.13 and statistical significant at high confidence with a logrank test p-value of 1.2 10⁻²⁸. In FIG. 11E, the second set of weights was used to stratify the COG high-risk cohort into 138 patients having a favourable prognosis and 110 patients having an unfavourable prognosis. In this case, the relative risk (hazard ratio) is 2.00 and a logrank p-value of 7.7 10⁻⁶.

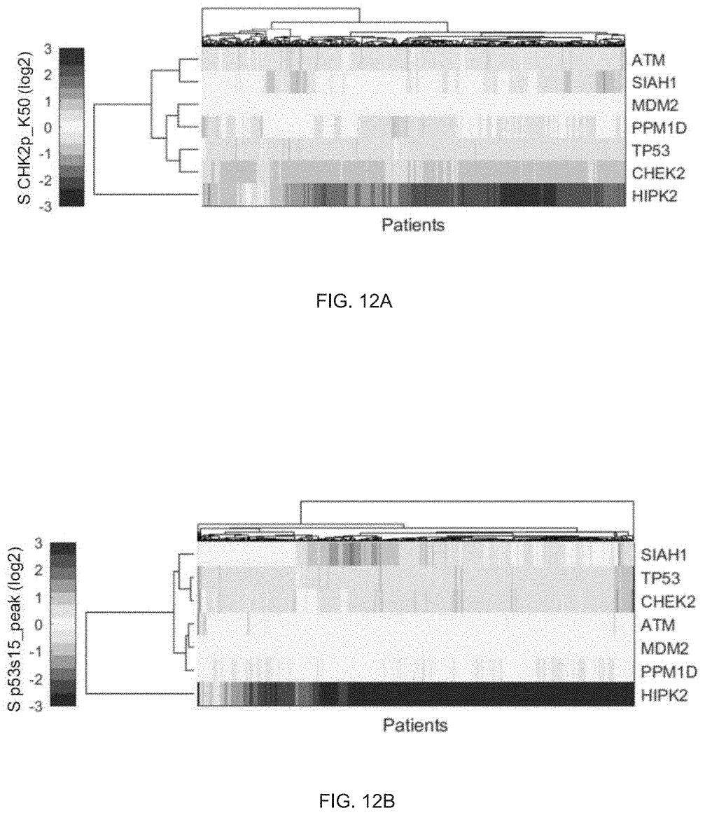

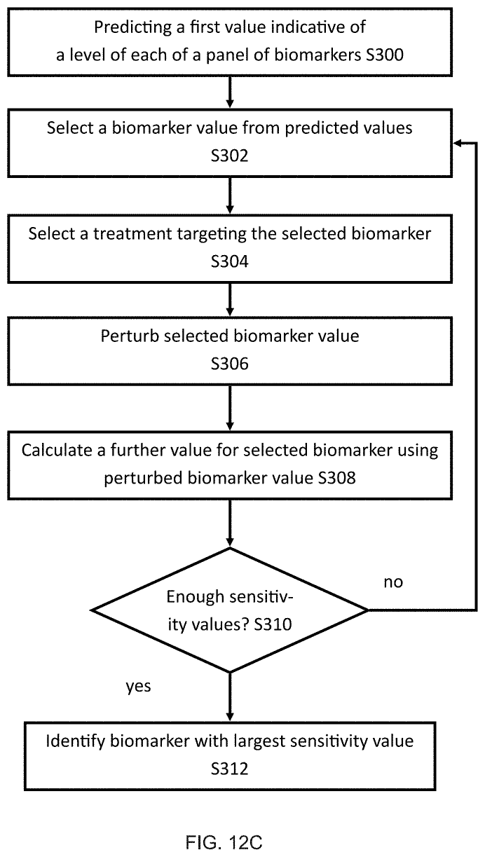

FIGS. 12A and 12B show the sensitivity analyses for different genes and proteins that control the prognostic features in the model. These have been shown to differ for different patients. For example, the table below the number of patients for which a putative drug target exerts the most control over the prognostic feature. The table shows that HIPK2 is a good target for most, but not all, patients. Such drugs could be used in combination with other therapies to maximise the benefit for the patient.

| Sensitivity | ATM | CHEK2 | HIPK2 | MDM2 | PPM1D | SIAH1 | TP53 |
|---|---|---|---|---|---|---|---|
| p53s15 | 3 | 6 | 675 | 0 | 1 | 0 | 3 |
| (peak) | | | | | | | |
| CHK2 | 1 | 60 | 610 | 0 | 8 | 9 | 0 |
| (K50) | | | | | | | |

FIG. 12C shows a method of analysing sensitivity. For example, the sensitivity analysis may be performed by using the model above to predict a first value indicative of a level of each of a panel of biomarkers (step S300). Then the method may comprise selecting a biomarker (e.g. ATM, CHEK2 etc) from the plurality of biomarkers (step S302). Next a treatment which targets the selected biomarker is selected (step S304). As defined above, the model comprises a plurality of parameters and the effect of the selected treatment on the selected biomarker may be included in the model by perturbing the parameter value corresponding to the selected biomarker in the model, for instance using a 1-fold change (multiply the parameter value by 2) (step S306). The model may then be used to calculate a sensitivity value for the selected biomarker by determining two output values for another biomarker within the model, e.g. by determining the p53s15 peak output value or the CHK2 half-activation output value, using both the original model and the perturbed model (step S308). Other biomarker outputs may be used as described above.

The sensitivity value may be calculated in a variety of suitable methods. For example, the sensitivity value may be calculating by comparing the simulated output of the unperturbed and perturbed model (i.e. first and second output values). The comparison may include calculating the difference or the ratio between the perturbed and unperturbed simulation output. For example:

$$S = \frac{{y}_{pertubed}}{{y}_{unperturbed}} ,$$

where S denotes the sensitivity value and y the simulated model output.

Alternatively, the sensitivity value may be calculated using:

$$S = \frac{{y}_{pertubed}}{{y}_{unperturbed}} \frac{{p}_{unperturbed}}{{p}_{perturbed}} , \\ or$$

$$S = \frac{{y}_{pertubed} - {y}_{unperturbed}}{{p}_{perturbed} - {p}_{unperturbed}} ,$$

where p denotes the parameter value of the targeted biomarker. Alternatively, the sensitivity value may be determined as described in the Fey 2015 Science Signaling paper above.

Once a sensitivity value has been calculated, the process may be repeated for further biomarkers to calculate a plurality of sensitivity values. In other words, there may be a determination as to whether the sensitivity value for all or a sufficient number of the biomarkers have been calculated (step S310) and if not the process repeats. the selected biomarker having a largest sensitivity value in the plurality of sensitivity values may be selected as the target for treatment (step S312). For example, as shown in the table above HIPK2 has the highest sensitivity for many patients.

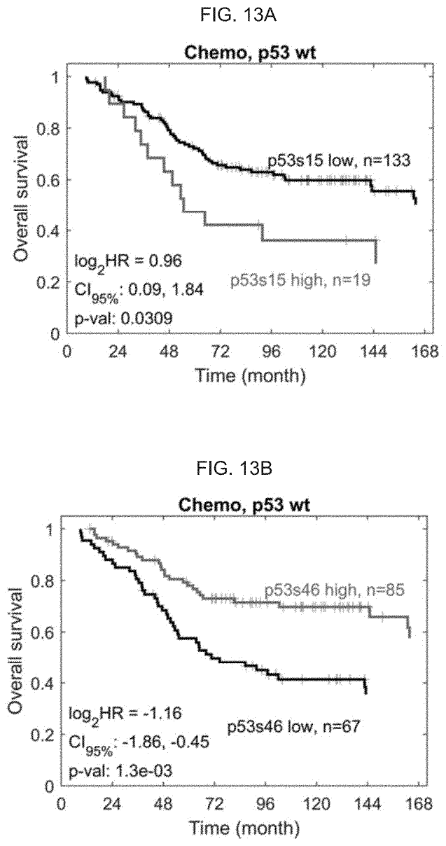

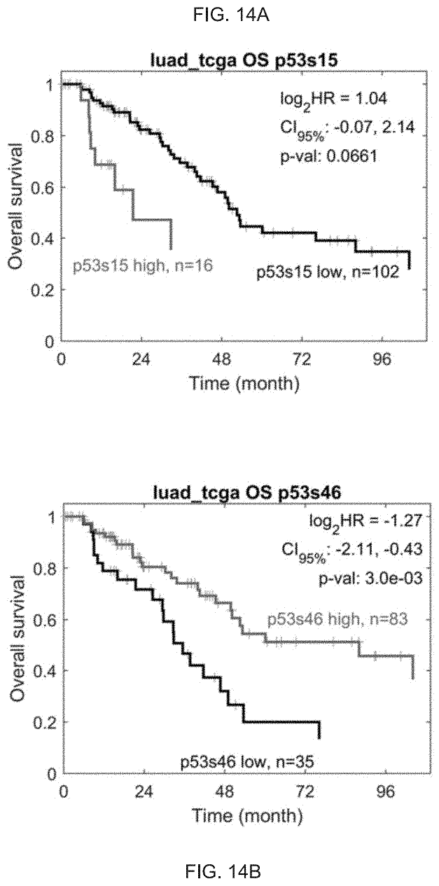

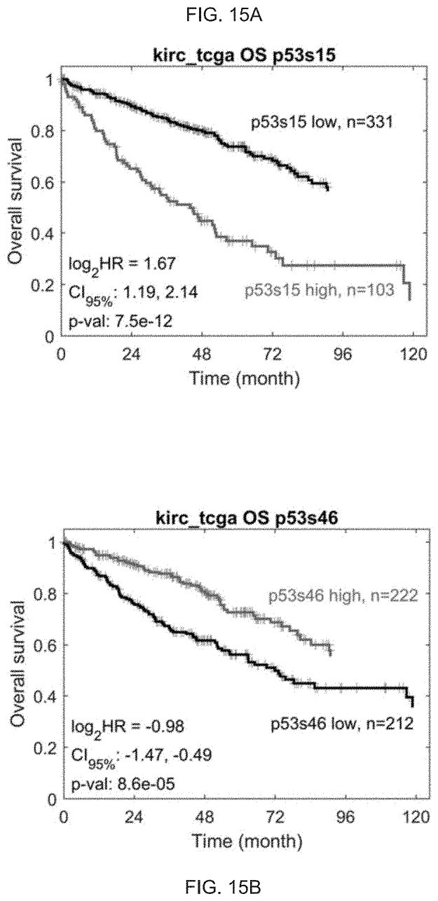

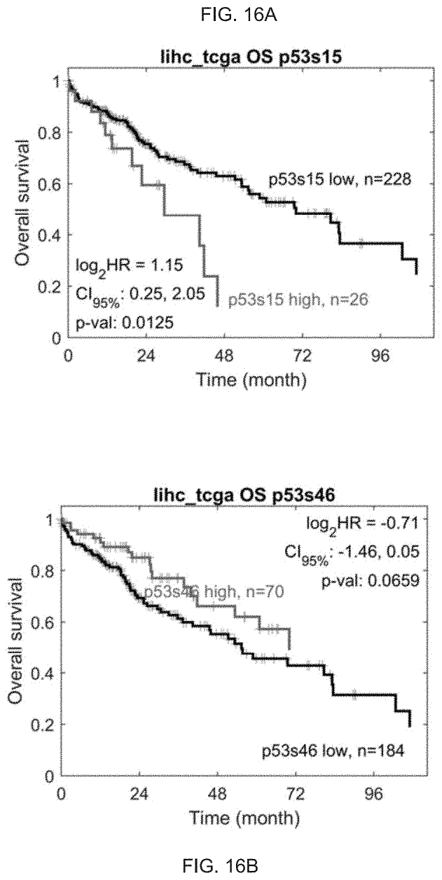

FIGS. 13A to 16B show various applications of the method show in FIG. 12C and described above. FIGS. 13A and 13B use a dataset of patients with breast cancer taken from the Molecular Taxonomy of Breast Cancer International Consortium (METABRIC), for example as described in “The Somatic Mutation Profiles of 2,433 breast cancers refines their genomic and transcriptomic landscapes” by Pereira et al published in Nat Commun 2016; 7:11479 doi:10.1038/ncomms11479. FIGS. 14A and 14B use a dataset of patients having lung cancer taken from the Cancer Genome Atlas Lung Adenocarcinoma (TCGA-LUAD) data collection. FIGS. 15A and 15B use a dataset of patients having kidney renal clear cell carcinoma from the Cancer Genome Atlas Kidney Renal Clear Cell Carcinoma (TCGA-KIRC) data collection. FIGS. 16A and 16B use a dataset of patients having liver hepatocellular carcinoma from The Cancer Genome Atlas Liver Hepatocellular Carcinoma (TCGA-LIHC) data collection. For each study (LUAD, KIRC, LIHC), the data was downloaded from the cbioportal on 11 Oct. 2019. There are also associated papers for each study but the data portal may contain more data. The associated papers are “Comprehensive Molecular Profiling of lung adenocarcinoma” (LUAD) published in Nature 2014 Jul. 31; 511(7511):543-50. doi: 10.1038/nature13385, “Comprehensive Molecular Characterisation of Clear Cell Renal Cancer” (KIRC) by Creighton et al published in Nature 2013, Jul. 4; 499(7456):43-9. doi: 10.1038/nature12222 and “Comprehensive and Integrative Genomic Characterisation of Hepatocellular Cancer” (LIHC) by Ally et al published in Cell 2017, Jun. 15; 169(7):1327-1341.e23. doi: 10.1016/j.cell.2017.05.046.

The table below shows the hazard ratio, confidence interval and p-value from the logrank test for each of FIGS. 13A to 16B. The results with the lowest p-value are statistically the best biomarkers.

| | Log₂ Hazard | Confidence | |
|---|---|---|---|
| Figure | Ratio | Interval (95%) | p-value |
| 13A | 0.96 | 0.09, 1.84 | 0.0309 |
| 13B | −1.16 | −1.86, −0.45 | 1.3e⁻⁰³ |
| 14A | 1.04 | −0.07, 2.14 | 0.0661 |
| 14B | −1.27 | −2.11, −0.43 | 3.0e⁻⁰³ |
| 15A | 1.67 | 1.19, 2.14 | 7.5e⁻¹² |
| 15B | −0.98 | −1.47, −0.49 | 8.6e⁻⁰⁵ |
| 16A | 1.15 | 0.25, 2.05 | 0.0125 |
| 16B | −0.71 | −1.46, 0.05 | 0.0659 |

FIGS. 13A and 13B stratify a group of 152 patients having breast cancer each of whom received chemotherapy, are p53 wild type patients described in the METABRIC database and for whom survival data is available. FIG. 13A stratifies the group of 152 patients into 133 patients with favourable prognosis and 19 patients with unfavourable prognosis based on their individual peak value for p53s15 using a p53s15_peak threshold. FIG. 13B stratifies the same group of patients into 85 patients with a favourable prognosis and 67 patients with an unfavourable prognosis based on their individual peak value for p53s46 using a p53s46_peak threshold. The results shown in FIG. 13B are better because of a lower p_value and a higher absolute log 2 hazard ratio. In other words, in this example, p53s46 is a better biomarker for stratifying the group.

FIGS. 14A and 14B stratify a group of 118 patients having lung cancer each of whom are p53 wild type patients described in the TCGA-LUAD database and for whom survival data is available. FIG. 14A stratifies the group of 118 patients into 102 patients with favourable prognosis and 16 patients with unfavourable prognosis based on their individual peak value for p53s15 using a p53s15_peak threshold. FIG. 14B stratifies the same group of patients into 83 patients with a favourable prognosis and 35 patients with an unfavourable prognosis based on their individual peak value for p53s46 using a p53s46_peak threshold. The results shown in FIG. 14B are better because of a lower p_value and a higher absolute log 2 hazard ratio. In other words, in this example, p53s46 is a better biomarker for stratifying the group.

FIGS. 15A and 15B stratify a group of 434 patients having renal cancer each of whom are p53 wild type patients described in the TCGA-KIRC database and for whom survival data is available. FIG. 15A stratifies the group of 434 patients into 331 patients with favourable prognosis and 103 patients with unfavourable prognosis based on their individual peak value for p53s15 using a p53s15_peak threshold. FIG. 15B stratifies the same group of patients into 222 patients with a favourable prognosis and 212 patients with an unfavourable prognosis based on their individual peak value for p53s46 using a p53s46_peak threshold. The results shown in FIG. 15A are better because of a lower p_value and a higher absolute log 2 hazard ratio. In other words, in this example, p53s15 is a better biomarker for stratifying the group.

FIGS. 16A and 16B stratify a group of 254 patients having liver cancer each of whom are p53 wild type patients described in the TCGA-LIHC database and for whom survival data is available. FIG. 16A stratifies the group of 254 patients into 228 patients with favourable prognosis and 26 patients with unfavourable prognosis based on their individual peak value for p53s15 using a p53s15_peak threshold. FIG. 16B stratifies the same group of patients into 70 patients with a favourable prognosis and 184 patients with an unfavourable prognosis based on their individual peak value for p53s46 using a p53s46_peak threshold. The results shown in FIG. 16A are better because of a lower p_value and a higher absolute log 2 hazard ratio. In other words, in this example, p53s15 is a better biomarker for stratifying the group.

As described above, the model describes patient-specific pathogenetic mechanisms which may be used in prognosis and treatment of patients. For example, the use of the model as described above may allow a clinician to know how well each patient responds to chemotherapy and how aggressively they should be treated which is useful. In particular, non-responders could be started immediately on immunotherapy, which is currently second line treatment. Unfortunately, current diagnostics do not allow this fine stratification.

The model described above is an improvement over the prior art models such as Zhang because these prior art models did not attempt to explain quantitative differences between different cells-types or patients. Furthermore, whilst such prior art models may consider biologic mechanisms, they are not compliant with the patient-specific modelling framework and did not contain patient-specific parameters. Finally, there is no teaching in Zhang of calibration using parameter estimation (i.e. no quantitative measurements from laboratory experiments were used).

At least some of the example embodiments described herein may be constructed, partially or wholly, using dedicated special-purpose hardware. Terms such as ‘component’, ‘module’ or ‘unit’ used herein may include, but are not limited to, a hardware device, such as circuitry in the form of discrete or integrated components, a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

What is claimed is:

1. A method for predicting whether a patient with cancer is likely to respond to a particular treatment, the method comprising:
applying a model comprising a set of coupled ordinary differential equations defining the rate of change of a plurality of biomarkers to predict a plurality of output values for each of the biomarkers for the treatment;
selecting a biomarker from the plurality of biomarkers;
comparing the output value for the selected biomarker to an associated threshold value for the selected biomarker; and
when the output value for the selected biomarker is below the associated threshold value, determining that the patient is likely to respond to the treatment;
wherein the plurality of biomarkers include p53, ATM, CHK2, SIAH1, HIPK2, WIP1 and MDM2.

2. The method of claim 1, wherein the plurality of biomarkers comprise unphosphorylated and phosphorylated forms of at least one of ATM, p53, SIAH1, WSB1, CHK1 and CHK2.

3. The method of claim 2, wherein the phosphorylated forms of p53 include pro-apoptotic residues such as S46 and cell-cycle arrest residues such as S15.

4. The method of claim 1, wherein the plurality of biomarkers comprise mRNA amounts for at least one of p53, WIP1, MDM2, MDM4 and MDMX.

5. The method of claim 1, wherein the plurality of biomarkers comprise protein amounts for at least one of HIPK2, WIP1, MDM2, MDM4 and MDMX.

6. The method of claim 1, comprising selecting the biomarker from a phosphorylated form of p53 at cell-cycle arrest residues such as S15, a phosphorylated form of ATM, a phosphorylated form of CHK2, a phosphorylated form of SIAH1, HIPK2, WIP1 and MDM2.

7. The method of claim 6, comprising comparing at least one of a peak value for the phosphorylated form of p53 at cell-cycle arrest residues such as S15, a peak value for the phosphorylated form of ATM, a peak value for the phosphorylated form of CHK2, a peak value for the phosphorylated form of SIAH1, a half activation value for HIPK2, a half activation value for WIP1 and a half activation value for MDM2, an amplitude value for HIPK2, an amplitude value for WIP1 and an amplitude value for MDM2.

8. The method of claim 1, wherein applying the model comprising predicting at least one of a peak value, an amplitude value and a half-activation value for each biomarker.

9. The method of claim 1, further comprising applying the treatment when it is determined that the patient is likely to respond to the treatment.

10. The method of claim 1, further comprising when it is determined that the total value is equal to or above the threshold, determining that the patient is not likely to respond to the treatment and determining an alternative treatment.

11. The method of claim 1, wherein the cancer is selected from the group consisting of neuroblastoma, breast cancer, lung adenocarcinoma, kidney renal clear cell carcinoma and liver hepatocellular carcinoma.

12. The method of claim 1, wherein the treatment is chemotherapy.

13. The method of claim 1, comprising using a set of training data to determine the associated thresholds for each biomarker value.

14. The method of claim 13, further comprising using a Cox regression analysis to determine the associated thresholds.

15. The method of claim 1, further comprising
providing a sample from the patient;
measuring a value indicative of a level of each of the biomarkers ATM, CHEK2, TP53, MDM2, PPM1D, SIAH1, HIPK2 and WSB1 and/or any of their paralogs, isoforms, or genes with similar biological functions within the sample; and
personalising the model to the patient by incorporating the measured values.

16. The method of claim 15, wherein personalising the model comprises defining patient-specific parameters for each biomarker.

17. The method of claim 16, wherein the patient-specific parameters comprise at least one of a value, ATMtot, for the protein expression of the gene ATM, a value, CHK2tot, for the protein expression of the protein CHK2, a value, SIAH1tot, for the protein expression of either of the genes SIAH1 and WSB1, a basal synthesis parameter, ksp530, for p53 mRNA, a basal synthesis parameter, ksmdm20, for MDM2 mRNA, a basal synthesis parameter, kswip10, for the gene WIP1 and a rate parameter, kshipk2, for HIPK2 translation.

18. The method of claim 1, further comprising using a set of training data to determine any parameters within the model.

19. A method for predicting whether a patient with cancer is likely to respond to a particular treatment, the method comprising:
measuring a value indicative of a level of each of the biomarkers ATM, CHEK2, TP53, MDM2, PPM1D, SIAH1, HIPK2 and WSB1 and/or any of their paralogs, isoforms, or genes with similar biological functions within a sample from the patient;
calculating a total value using a weighted sum of the measured values; wherein each biomarker value has an associated weight;
comparing the total value to a threshold value and
when it is determined that the total value is below the threshold value, determining that the patient is likely to respond to the treatment.

20. The method of claim 19, further comprising using a set of training data to determine the associated weights for each biomarker value.

21. The method of claim 20, further comprising using a Cox regression analysis to determine the associated weights.

22. The method of claim 19, further comprising applying the treatment when it is determined that the patient is likely to respond to the treatment.

23. The method of claim 19, further comprising when it is determined that the total value is equal to or above the threshold, determining that the patient is not likely to respond to the treatment and determining an alternative treatment.

24. The method of claim 19, wherein the cancer is neuroblastoma, breast cancer, lung adenocarcinoma, kidney renal clear cell carcinoma or liver hepatocellular carcinoma.

25. The method of claim 19, wherein the gene expression of the biomarkers is measured.

26. A method for predicting whether a patient with cancer is likely to respond to a particular treatment, the method comprising:
applying a model comprising a set of coupled ordinary differential equations defining the rate of change of a plurality of biomarkers to calculate a first output value for a biomarker in the plurality of biomarkers, wherein the model comprises a plurality of parameter values associated with the plurality of biomarkers;
selecting a biomarker from the plurality of biomarkers;
selecting a treatment which targets the selected biomarker;
perturbing the parameter value corresponding to the selected biomarker in the model;
applying the model using the perturbed parameter value to calculate a second output value for the biomarker within the plurality of biomarkers,
comparing the first and second output values to derive a sensitivity value for the selected biomarker;
iterating the selecting and calculating steps for further biomarkers to calculate a plurality of sensitivity values; and
identifying the selected biomarker having a largest sensitivity value in the plurality of sensitivity values.

27. The method of claim 26, wherein the sensitivity value is derived by calculating at least one of the difference and the ratio between the first and second output values.

28. The method of claim 27, further comprising applying the treatment which targets the identified biomarker.

29. A kit comprising reagents that specifically bind to each member of a panel of biomarkers consisting of ATM, CHEK2, TP53, MDM2, PPM1D, SIAH1, HIPK2 and WSB1 or their proteins and/or any of their paralogs, isoforms, or genes with similar biological functions.

30. A kit according to claim 29, wherein the reagents are PCR primer sets.
